(19) United States
(12) Patent Application Publication
(10) Pub. No.: US 2026/0176670 A1
(43) Pub. Date: Jun. 25, 2026

(54) SYSTEMS, COMPOSITIONS, AND METHODS FOR SINGLE CELL ANALYSIS

(71) Applicant: Fluid Discovery, San Francisco, CA (US)

(72) Inventor: Adam Abate, San Francisco, CA (US)

(21) Appl. No.: 19/127,236

(22) PCT Filed: Nov. 3, 2023
(86) PCT No.: PCT/US23/36757

§ 371 Date: May 5, 2025

Related U.S. Application Data
(60) Provisional application No. 63/382,353, filed on Nov. 4, 2022.

(51) Int. Cl. C12Q 1/6806; C12Q 1/6851; C12Q 1/6869

(52) U.S. Cl. CPC C12Q 1/6806; C12Q 1/6851; C12Q 1/6869

(57) ABSTRACT

Provided herein are systems, compositions, and methods for single cell analysis. In particular, provided herein are systems, compositions, and methods for scalable, high-throughput isolation and sequencing of nucleic acids from rare and/or fragile single cells.

### FIELD

Provided herein are systems, compositions, and methods for single cell analysis. In particular, provided herein are systems, compositions, and methods for scalable, high-throughput isolation and sequencing of nucleic acids from rare and/or fragile single cells.

### BACKGROUND

Rare cells play an important role in many biological processes and diseases but are understudied due to the challenge of isolating and analyzing them in sufficient numbers. This problem is exemplified by the brain. Mammalian brains are the seat of many scientific mysteries, including the evolution of consciousness and uniquely human traits like speech and sapience. Human brains are also one of the most complex organs to have evolved, comprising over 100 billion neurons and 100 trillion connections. An important tool in the study of any complex organ system is construction of “single cell atlases” that characterize the genomic properties of all cell types. Atlasing projects have benefited from advances in single cell genomics, which have increased the number of cells that can be sequenced from 10s a decade ago to 10s of 1000s today. Despite these advances, the throughput of conventional single cell methods remains far below that necessary to generate complete maps of even the simplest mammalian brains, comprising a significant barrier to understanding the roles of rare cell types in diseases of the aging brain. For example, Von Economo neurons (VENs) are unique to a small number of intelligent animals and are important to social emotions and the pathophysiology of neuropsychiatric disorders including frontotemporal dementia, schizophrenia, and autism. However, VENs comprise ˜1.25% of neurons in the human anterior cingulate cortex and, thus, are poorly represented in existing atlases. Attempts to isolate and sequence VENs by fluorescence activated cell sorting (FACS) are hindered by the lack of effective antibodies for labeling, while brute-force application of single cell approaches fails to capture VENs in sufficient numbers for statistically powerful characterization. Thus, current brain atlases have critical gaps resulting from the inability to target rare cell types. Improved technologies are needed.

### SUMMARY

Provided herein are systems, compositions, and methods for single cell analysis. In particular, provided herein are systems, compositions, and methods for scalable, high-throughput isolation and sequencing of nucleic acids from rare and/or fragile single cells.

The systems, compositions, and methods provided herein facilitate analysis of rare and/or fragile cells that are important for understanding and intervening in biological processes such as brain aging. In particular, provided herein are systems, compositions, and methods for isolating extremely rare cells based on nucleic acid biomarkers. The ability to sort based on nucleic acids, rather than proteins, is opportune because biomarkers of rare cell types critical to diverse biological processes including, for example, brain aging may be RNAs with no known protein counterparts. In turn, approaches based on in situ hybridization are insensitive and interfere with subsequent single cell sequencing. To support sensitive and accurate detection of rare cells, the technology, in some embodiments, comprises digital droplet PCR, with single molecule sensitivity that surpassing that of in situ hybridization. Using mRNA biomarkers instead of surface proteins not only unlocks sorting of nuclei, but further expands biomarkers to any nuclear RNA. This approach increases the power to identify and characterize cell types that are rare or difficult to enrich due to limited surface markers. In some embodiments, to sequence the sorted cells, the technology employs a droplet barcoding technology (e.g., PIPseq).

In some embodiments, the present invention provides a combination of a sorting method, (e.g., PCR activated cell sorting (PACS)) and a sequencing method (e.g., pre-templated instant partitions sequencing (PIPseq)) that supports microfluidic-free encapsulation, lysis, and barcoding of rare cells in large numbers. By leveraging these technologies, 10s of 1,000,000s of cells may be sorted to target 100s of 1000s of rare cells of interest for single cell sequencing.

Additionally, PIPseq is compatible with nuclear staining and sorting approaches, facilitating analysis of fresh frozen human samples and enrichment of desired rare cells lacking sortable protein surface markers. Accordingly, in some embodiments, the present invention overcomes major barriers faced by atlasing and other projects that target single cell and nuclei isolation and characterization comprising broad, unbiased surveys of cellular diversity, and targeted deep characterization of all cells including rare cells of particular interest.

The importance of single cell genomics has led to numerous academic and commercial systems. Most are based on wells (Smart-seq, MARS-seq, Seq-Well)³⁶⁻³⁸, microfluidic droplets (Drop-seq, InDrops, 10x Genomics)^{19,20,22}, or combinatorial indexing (SCI-seq)³⁹, many of which are now offered commercially. Major vendors are Honeycomb⁴⁰, Parse⁴¹ and 10X Genomics²².

Of these, only combinatorial indexing and droplet microfluidics are competitive with PIPseq in scalability to large cell numbers. Like PIPseq, SCI-seq is a “bulk” approach that uses no microfluidics and processes all cells in a single reaction vessel. Thus, it can be conducted via bulk fluid manipulation in well plates, and is extremely scalable—like PIPseq, to the tens of millions of cells³¹. However, combinatorial indexing has significant drawbacks that limit its utility. The indexing workflow requires multiple rounds of split-pool mixing on hundreds of wells.^{39.42} This is complex and laborious, and sample contamination and loss are major constraints due to extensive handling. Droplet microfluidic barcoding (10X Genomics), by contrast, provides the high data quality⁴³ because the molecular biology is independent of the compartmentalization process, and can therefore be flexibly optimized^{20,21,44} However, the requirement of microfluidics greatly adds complexity and cost, and severely restricts the number of cells that can be barcoded. Additionally, microfluidic channels are prone to clogging, especially with large or extended cell types like neurons. As well, neuronal cells clump while awaiting encapsulation thereby resulting in mixed cell data. With PIPseq clogging is impossible because cells and nuclei are never flowed through microfluidic channels, and clumping is minimized because encapsulation occurs in under a minute. The result is that PIPseq provides superior neuronal cell data compared to the 10X Chromium. Moreover, 10X Chromium is difficult to integrate with FACS. Sorted cells must be transferred, resuspended, and be re-quantitated before being introduced into the microfluidic chip. These steps add time and handling that perturbs gene expression and reduces data quality. Cells are lost with each pipetting step such that, for rare cells, it is common to not have enough cells to load into the microfluidic chip. Accordingly, PIPseq is a leap forward because it combines the speed and scalability of bulk processing encapsulation with the optimal molecular biology of droplets. Removal of microfluidics simplifies workflows and automation, making it superior for analyzing large numbers of samples in well plates. PIPseq is cost-effective because no instrument is required, and contamination is minimized because the reaction occurs in a sealed tube or well without sample transfer³¹. As a result, while all other methods require >500 input cells, PIPseq supports barcoding from tens of cells to tens of millions, yielding a dynamic range larger than any competitor³¹. Moreover, the simplicity of the approach and workflow make it simple to integrate with FACS, allowing sorted cells to be directly dispensed into the PIPseq reaction tube, where they are lysed and barcoded without further handling. This is critical to obtaining the highest quality gene expression data and minimizing loss of rare cells recovered from limited brain samples. Thus, in some embodiments, the present invention combines the sorting capabilities of scRNA-seq (PIPseq) with targeted sorting based on nucleic acid biomarkers (PACS)⁴⁵ to provide high-resolution mapping of cells (e.g., brain cells) and detection of rare cell types. The technology finds use, for example, to define the transcriptional properties of rare human layer 5 cortex VENs and other cell types with relevance to neuropsychiatric disorders and brain aging^{3,7}.

A challenge in current brain atlasing surveys is the inability to enrich specific cell types for in-depth analysis. FACS enriches cell types with surface protein biomarkers, but this approach requires specific antibodies, which do not always mark cell types of interest. Moreover, direct sorting of brain cells is rarely possible, as cleanly dissociating brain tissue stresses cells, perturbs gene expression, and produces low-quality RNAseq data³². Consequently, large scale atlasing surveys rely on nuclei sequencing because they can be quickly and cleanly extracted from brain tissue and are compatible with single cell (nuclei) sequencing²¹. Nuclear isolation, however, removes the cell membrane, precluding sorting via FACS detected surface markers, a major problem for rare brain cell isolation. Fluorescence in situ hybridization labels mRNA transcripts and therefore affords a route towards recovering the nuclei of specific cells (FISH-FACS). However, the approach is difficult to integrate with single cell sequencing for several reasons. The fixation, permeabilization, and staining degrades mRNA quality, and the multiple washing steps result in cell clumping and loss, such that insufficient nuclei are recovered for single cell barcoding. Additionally, in situ hybridization methods that are compatible with FACS are insensitive, often missing low or moderately expressed transcripts. The result is that data quality is poor, and the method is rarely used for rare cell applications³³. For example, in prior experiments performed to study VENs, L5 neurons sorted from brain tissue captured only 23 nuclei, of which many were off-target fork cells and L5 pyramidal neurons⁷. Markers specific to L5 pyramidal neurons or VENs (ADRA1A, GABRQ, and ITGA4) are not nuclear proteins and, thus, cannot be used for antibody-based sorting. What is needed is an effective way to isolate and deeply sequence important rare brain and other tissue cell types.

Conventional microfluidic techniques encapsulate cells serially (i.e., one-by-one) by loading them into water-in-oil droplets⁴⁶. Throughput is proportional to the speed of the microfluidic device, such that increasing cell numbers requires running devices longer. Consequently, to run commercial systems (e.g., 10X Genomics) at the scale of a million cells requires hours of droplet generation and >$150,000^{26,28,47} in microfluidic kits and reagents. Technologies of the present invention that rapidly process large numbers of cells in minutes at reduced cost greatly increase the ability of atlas projects to survey cellular diversity. The present invention leverages droplet self-assembly for scalable scRNAseq. The technology uses barcoded Pre-templated Instant Partitions (PIPs) to perform cell capture, lysis, and mRNA recovery in minutes using common equipment³¹. With PIPseq, the number of droplets scales with container volume, not run time, because droplet generation occurs in parallel via self-assembly. Thus, increasing the cells barcoded from 10,000 to 100,000 requires increasing the tube volume from ˜10 to 100 microliters with the same emulsification time (˜1 min)³¹. Accordingly, barcoding 10 million cells can be accomplished in a 10 mL PIP reaction, and 100 million cells in the same tube size by implementing hashing (i.e., oligonucleotide-tagged antibodies)^{48,49}. This scalability is required to match expected increases in sequencing capacity, which should more than quadruple in the next five years⁵⁰. At this rate, sequencing 10 million single cell transcriptomes should be possible for ˜$100,000, and 100 million cells in about a decade. This is sufficient to sequence every cell in most organs of the mouse, and sizable portions of the human brain¹⁰⁻¹⁴ and will provide tissue atlases at unprecedented scale. PIPseq provides a technical leap, aligning the bottleneck from cell preparation as it is today to sequencing, and keeping pace with expected increases in sequencing capacity over the coming decade. Compared to combinatorial indexing that requires hundreds of pipetting operations between wells in plates and expensive instrumentation for automation, PIPseq's workflow is rapid, facile, and may be performed at the bench to barcode the same number of samples and cells. No microfluidics or other specialized equipment is required beyond a vortexer and thermal cycler. In addition to much lower cost, the workflow is fast and flexible, allowing cells to be encapsulated, lysed, and mRNA captured with ˜10 min of hands-on time, and thereby implemented in conditions in which instrument processing is challenging, like BSL-3 and 4 laboratories. In such cases, using shared instruments in core facilities or custom workflows (Drop-seq) is a major logistical challenge. A unique and valuable feature of PIPseq is its compatibility with well plates. Since encapsulation is performed with a vortexer, the system is portable to projects that require low cell inputs (rare cells harvested by microdissection or sorting) and high sample numbers (cells harvested over developmental time or brain region). Thus, PIPseq is scalable in cell (100-10M) and sample number (1-100s) across a large range of inputs.

In some embodiments, the present invention provides a method for processing cells or nuclei, comprising: a) individually encapsulating a plurality of cells or nuclei from a sample by forming a plurality of water in oil droplets in a reaction vessel, wherein the droplets comprise a bead affixed to a nucleic acid capture molecule; b) lysing the cells or nuclei and capturing RNA from the cells or nuclei with the nucleic acid capture molecule; c) removing beads with captured RNA from the droplets and reverse transcribing captured RNA; d) re-encapsulating the beads to form amplification droplets; e) amplifying nucleic acid in the amplification droplets to generate amplified nucleic acid; f) removing amplified nucleic acid from the amplification droplets; and g) isolating amplified nucleic acid derived from the captured RNA by automated sorting.

In some embodiments, the cells comprise rare cells. In some embodiments, the nuclei comprise nuclei derived from rare cells. In some embodiments, the sample is a brain tissue sample. In some embodiments, the droplets further comprise a lysis reagent. In some embodiments, reaction vessel comprises a reaction tube. In some embodiments, the forming comprises agitating (e.g., vortexing). In some embodiments, the capture molecule comprises a barcode. In some embodiments, the capture molecule comprises a cDNA sequence complementary to the RNA. In some embodiments, the cDNA is complementary to an RNA expressed in a rare cell.

In some embodiments, the method comprises a plurality of different nucleic acid capture molecules affixed to one or more beads. In some embodiments, the plurality of different nucleic acid capture molecules comprises a plurality of different cDNA sequences. In some embodiments, the plurality of different cDNA sequences is complementary to a plurality of different RNA molecules that are each expressed in a rare cell. In some embodiments, the plurality of different RNA molecules is uniquely expressed in the rare cell.

In some embodiments, the amplifying comprises PCR. In some embodiments, the PCR comprises fluorescent amplicon tethering ddPCR. In some embodiments, the isolating comprises FACS.

In some embodiments, the method further comprises the step of h) analyzing the amplified nucleic acid derived from the captured RNA. In some embodiments, the analyzing comprises sequencing the amplified nucleic acid derived from the captured RNA.

In some embodiments, the droplets further comprise a heat activated lysis reagent wherein the lysing comprises heating the droplets.

In some embodiments, the present invention provides a kit comprising one or more components useful, necessary, and/or sufficient to practice methods of the present invention.

In some embodiments, the kit comprises one or more or each of: a) beads, b) nucleic acid capture molecules, c) oil, d) water, e) lysing reagent, f) one or more reverse transcription reagents, g) one or more nucleic acid amplification reagents, h) one or more positive control reagents, i) one or more negative control reagents, j) an agitation instrument, k) a sorting instrument, l) one or more nucleic acid sequencing reagents, m) software, n) instructions, and o) a reaction vessel. In some embodiments, the nucleic acid capture molecules are affixed to the beads. In some embodiments, the nucleic acid capture molecules comprise one or more or each of: a) a barcode sequence, b) a poly-T sequence, c) a sequence complementary to the RNA, and d) a UMI sequence. In some embodiments, the lysing reagent is a heat activated lysing reagent. In some embodiments, the reverse transcription reagents comprise one or more or each of: a) a reverse transcriptase, b) a primer, c) an RNase inhibitor, d) dNTPs, e) a buffer, and f) a divalent cation. In some embodiments, the nucleic acid amplification reagents comprise one or more or each of: a) a DNA polymerase, b) one or more primers, c) dNTPs, d) a buffer, e) a label, and f) a divalent cation. In some embodiments, the label comprises a fluorescent label. In some embodiments, the fluorescent label comprises a label selected from the group comprising fluorescein and its derivatives, rhodamine and its derivatives, cyanine and its derivatives, coumarin and its derivatives, Cascade Blue and its derivatives, Lucifer Yellow and its derivatives, BOD IP Y and its derivatives, and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. In some embodiments, the label is attached to a primer. In some embodiments, the positive control reagents comprise one or more or each of: a) a cell, b) a nucleus, and c) an RNA. In some embodiments, the agitation instrument comprises a vortexing instrument. In some embodiments, the sorting instrument comprise a FACS instrument or a magnetic-activated cell sorting (MACS) instrument. In some embodiments, the nucleic acid sequencing reagents comprise one or more or each of: a) one or more primers, b) a DNA polymerase, c) library preparation reagents, d) dNTPs, e) one or more detectable labels, and f) a buffer. In some embodiments, the software comprises instructions for running on a computer processor that carry out one or more or each of the functions: a) operating the agitation instrument, b) operating the sorting instrument, c) heating or cooling an instrument, d) controlling a reverse transcription reaction, e) controlling an amplification reaction, f) controlling a sequencing reaction, g) collecting data, h) storing data, i) analyzing data, and j) reporting data. In some embodiments, the reaction vessel is a tube. In some embodiments, the tube is microcentrifuge tube or a test tube. In some embodiments, the reaction vessel is a flask.

In some embodiments, the present invention provides use of the kits of the present invention. In some embodiments, the present invention provides use of the kits of the present invention for processing a cell or nuclei. In some embodiments, the present invention provides use of the kits of the present invention for analyzing one or more rare cells or nucleic derived from rare cells.

In some embodiments, the present invention comprises a reaction mixture comprising a reaction present during a method of the present invention. In some embodiments, the reaction mixture is present during individually encapsulating a plurality of cells or nuclei from a sample by forming a plurality of water in oil droplets in a reaction vessel, wherein the droplets comprise a bead affixed to a nucleic acid capture molecule. In some embodiments, the reaction mixture is present during lysing the cells or nuclei and capturing RNA from the cells or nuclei with the nucleic acid capture molecule. In some embodiments, the reaction mixture is present during removing beads with captured RNA from the droplets and reverse transcribing captured RNA.

In some embodiments, the reaction mixture is present during re-encapsulating beads to form amplification droplets. In some embodiments, the reaction mixture is present during amplifying nucleic acid in the amplification droplets to generate amplified nucleic acid. In some embodiments, the reaction mixture is present during removing amplified nucleic acid from the amplification droplets. In some embodiments, the reaction mixture is present during isolating amplified nucleic acid derived from the captured RNA by automated sorting.

In some embodiments, the present invention provides methods for processing cells or nuclei, comprising one or more or each of the steps of: a) individually encapsulating a plurality of cells or nuclei from a sample by forming a plurality of water in oil droplets in a reaction vessel, wherein the droplets comprise a bead affixed to a nucleic acid capture molecule; b) lysing the cells or nuclei and capturing RNA from the cells or nuclei with the nucleic acid capture molecule; c) removing beads with captured RNA from the droplets and reverse transcribing captured RNA; d) re-encapsulating beads to form amplification droplets; e) amplifying nucleic acid in the amplification droplets to generate amplified nucleic acid; f) removing amplified nucleic acid from the amplification droplets; and g) isolating amplified nucleic acid derived from the captured RNA by automated sorting. In some embodiments, the amplifying is selected from the group comprising PCR, LAMP, NASBA, HAD, SDA, NEAR, SPIA, RCA and RPA amplifying.

### DEFINITIONS

As used herein, the term “sample” or “biological sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples.

### DETAILED DESCRIPTION

Provided herein are systems, compositions, and methods for single cell analysis. In particular, provided herein are systems, compositions, and methods for scalable, high-throughput isolation and sequencing of nucleic acids from rare and/or fragile single cells.

The technology finds use with any cell type. The technology is particularly suited to the isolation and analysis of rare and/or fragile cell types. Rare and/or fragile cell types include, but are not limited to, cells in the brain (e.g., pericytes, smooth muscle cells, microglia, and subtypes of vascular and leptomeningeal cells), tumor cells, stem cells, cancer stem cells, immune cells, infected cells, and the like.

The technology finds use for the analysis of cells for any purpose, including but not limited to, cell discovery, cell atlasing, basic research, drug discovery, disease screening, disease diagnostics, therapeutic companion diagnostics, therapy monitoring, health monitoring, study of metabolism, study of aging, and the like.

The technology is illustrated herein in the context of brain atlasing. This example is provided to exemplify the power and benefits of the technology. However, it should be understood that the technology is not limited to use with brain tissue or brain atlasing projects.

Brain atlasing projects characterize the cellular diversity of the human and mouse brains^{8,9}. Profiling the full extent of diversity, including cells of low abundance, requires sequencing many cells. A typical mouse brain has over a hundred million cells, the human brain over a hundred billion¹⁰⁻¹², and other important tissue and organ systems comprise cell numbers of this scale^{13,14}. While tissue atlasing has greatly benefited from rapid improvements in single cell methods¹⁵, the current scale of single cell sequencing remains far below necessary to meet the scope of ambitious atlasing efforts¹⁶⁻¹⁸. The most advanced technologies use high throughput microfluidics to compartmentalize and barcode mRNA¹⁹⁻²³, processing ˜10,000 cells per channel. While this represents a significant advance over what was possible only ten years ago²⁴, it is far below necessary to characterize the diversity of cells found in most tissues, including the brain^{12,14}. Moreover, scaling beyond millions of cells is difficult due to fundamental microfluidic limits, the requirement for and cost of microfluidic instrumentation, and barriers to automate them with robotics²⁵. Thus, while current sequencing capacity can analyze millions of cells, cell barcoding remains a bottleneck. For example, a modest atlas of ˜100,000 cells currently costs ˜$19,000 in microfluidic barcoding and ˜$9,000 in sequencing²⁶⁻²⁸. Moreover, with anticipated cost reductions due to the entry of new sequencing competitors on the market (Ultima, Element), sequencing prices are expected to drop by 10-fold in the next two years^{29,30}, exacerbating the disparity between prepping cells and sequencing them. Brute force scaling to the multimillion level, as needed to generate complete atlases of many brain structures is thus prohibitively costly. To advance atlasing studies and permit capture of important rare cell types, new methods for single cell sequencing are needed.

In some embodiments, the present invention provides a combination of a sorting method, (e.g., PACS) and a sequencing method (e.g., PIPseq) that supports microfluidic-free encapsulation, lysis, and barcoding of cells in large numbers. This combination is referred to herein as PIP-PACS. PIP-PACS leverages an efficient approach for sequencing large numbers of cells. In some embodiments, the approach employs pre-templated instant partitions (PIPs)³¹ (Hatori et al., Anal Chem. 2018; 90(16):9813-9820., herein incorporated by reference in its entirety), a method that barcodes millions of cells or nuclei in a tube without microfluidics. This allows rare cells to be isolated by FACS or PACS directly into the PIPseq tube without additional transfers, increasing the efficiency of cells captured. Moreover, because cells are lysed and barcoded within minutes of isolation, perturbations due to handling are minimized, yielding superior gene expression data. The technology provided herein allows robust capture of rare cells missed by current approaches.

Microfluidic approaches require unique instrumentation and consumables that are expensive and difficult to automate²⁵. By contrast, in some embodiments PIPseq barcodes cells in wells on a plate, requiring no transfer of reagents to microfluidic chips, and allows ready use of FACS for cell or nuclei pre-enrichment. Removal of microfluidics reduces consumable and labor costs while minimizing contamination and cell loss.

In some embodiments, the sorting method comprises nucleic acid cytometry based on specific nucleic acid biomarkers. In some embodiments, the technology uses droplet microfluidics to encapsulate cellular material in water droplets suspended in oil, creating discrete reactors. Each reactor comprising a cell may generate a signal that identifies the presence of a specific sequence in a cell. Positive droplets, identified by the signal, are sorted and their contents released for further analysis. Suitable approaches for sorting are described in Clark and Abate, Lab Chip. 2017; 17(12):2032-2045., herein incorporated by reference in its entirety.

Exemplary implementations of the technology are described in the Examples section below.

Work conducted in the course of development of the present invention has established high throughput methods for isolating and RNA sequencing cells based on nucleic acid biomarkers (PACS). In some embodiments, the technology provides the ability to capture transcriptomes from millions of single cells in parallel on beads or other solid supports, then sort the beads based on PCR detection of target biomarkers. Prior uses of capture have led to high-impact publications and commercial products (Mission Bio),^{34,35} but have limitations. First, they use microfluidics for the cell encapsulation and sorting. This reduces throughput and necessitates microfluidic expertise that impedes adoption by non-experts. Second, the captured single cell transcriptomes were not barcoded, requiring that each sorted cell be dispensed into a well plate for single cell sequencing. This limits the number of cells that can be sequenced, and adds significant workflow complexity and cost to the library preparation. In some embodiments, the present invention provides a fully microfluidic free process. PIPseq is used to barcode the cells using scalable PIP emulsification. With 50 μm PIPs, 10 million cells can be barcoded in a 5 mL reaction for ˜$1000 (0.01 cent/cell before sequencing), a hundredth the cost of microfluidic approaches. With flow cytometry, 100 million PIPs can be sorted in ˜2 hrs. The cells are lysed, and the transcriptome stabilized as cDNA at this point, such that sorting for this time will not degrade transcriptome data. Additionally, the ability to perform these steps without microfluidics and using common laboratory equipment lowers the barrier of adoption. A detailed protocol describes synthesizing suitable PIP particles is provided in Delley and Abate, Sci Rep. 2021 May 25; 11(1):10857., herein incorporated by reference in its entirety. Suitable particles may also be purchased from vendors including Fluent Biosciences and RAN Biotechnologies.

Application of the technology to the analysis of Von Economo cells demonstrates the power of the approach. Von Economo neurons (VENs) are rare and important to diseases of aging, but do not have antibody markers by which to efficiently detect, isolate, and sequence them. Consequently, VENs are underrepresented in existing brain atlases, and their function in diseases of aging is poorly understood. In some embodiments, the technology provided herein employs mRNA sorting methods to isolate and analyze Von Economo cells. In some embodiments, the technology targets genes specific to Von Economo cells (e.g., ADRAIA (adrenoceptor alpha 1A), GABRQ (Gamma-Aminobutyric Acid Type A Receptor Subunit Theta), VMAT2 (vesicular monoamine transporter 2), LYPD1(LY6/PLAUR Domain Containing 1), SULF2 (Sulfatase 2), CHST8 (Carbohydrate Sulfotransferase 8), and ITGA4 (integrin subunit alpha 4)). The sorting approach is generalizable to any cell type that can be defined by mRNA markers, applicable to tens of millions of cells, and directly compatible with single cell mRNA sequencing^{34,35}. These attributes provide for deep characterization of rare cell types currently underrepresented in brain atlases but known to play important roles to aging and disease.

VENs are among the most interesting cells in the brain due to unique features including their absence in experimental models; their presence in great apes, humans, whales, dolphins, and elephants only; their function in social behaviors and networks; their postnatal origins; their hemispheric lateralization which may relate to specialization of the right hemisphere for social emotion; and their implication in neurodevelopmental and neurodegenerative diseases³. In common with other cortical regions, the human anterior cingulate cortex (ACC) contains abundant and rare cell types, including excitatory and inhibitory neurons and numerous non-neural cells, and is one of only two brain regions containing VENs⁴. Among primates, VENs are found only in humans and great apes, and are thus a recently evolved cell type that arose in the hominid lineage in the last 15 million years⁵. Located in layer 5 of ACC and fronto-insular cortex, they have a unique developmental trajectory. Unlike most neurons that arise prenatally, VENs emerge largely after birth and increase through early childhood, either through delayed differentiation or migration from postnatal neurogenic regions⁶. Selective loss of VENs may contribute to neuropsychiatric disorders characterized by social-emotional deficits including frontotemporal dementia, schizophrenia, and autism³. VENs are a rare cell type, constituting ˜1.25% of neurons in human ACC. In a recent single cell transcriptomic study⁷ the authors used microdissection of fronto-insular cortex layer 5 with extraction of nuclei and FACS to enrich NEUN+ cells. Of 879 nuclei, the cluster containing VENs from human cortex comprised only 23 cells. Despite morphological diversity, the authors could not subcluster based on genomic differences, and thus concluded that the small sample size lacked statistical power to divide the cluster into known neuron subtypes⁷. In some embodiments, the present invention overcomes this limitation by using sorting modalities to enrich for these cells, sequencing ˜10,000 individual VEN nuclei per brain sample. Data of this depth supports detailed characterization of VEN phenotypes and analysis of their roles in brain biology and disease.

In some embodiments, the present invention supports deep transcriptional profiling of VEN cells with an easy-to-use method for rare cell enrichment comprising sorting cells based on RNA biomarkers. PIP emulsification is used to replace conventional microfluidic steps, thereby providing a simple and rapid version of PCR-activated cell sorting (PACS) technology^{31,58,59}. Using mRNAs, instead of surface proteins, not only unlocks sorting of nuclei, but also expands biomarkers to any nuclear RNA of any cell.

In some embodiments, assaying nuclei, rather than whole cells, is a pre-requisite for obtaining quality RNA-sequencing data from different brain tissues including lightly fixed and post-mortem samples. However, nuclei preparation methods remove the cell membrane, eliminating markers used for traditional methods like FACS (fluorescence activated cell sorting) and MACS (magnetic-activated cell sorting), and making targeted cell types determined by scRNA-seq difficult to enrich and study further. No commercial technologies are currently available for isolating nuclei based on their specific RNA-content, especially low-abundance transcripts. In some embodiments, the present invention leverages PIPs emulsification, and the molecular advances made to develop to PACS microfluidic workflow described herein, to build an RNA-biomarker sorting technology accessible to labs without microfluidic expertise. Moreover, barcoding of single cells during reverse transcription allows many cells to be pooled for sequencing. Because cells are defined by their DNA barcode, this barcode can be utilized to re-isolate and sequence (at deeper coverage) any cell of interest from the original dataset.

In some embodiments, the present invention provides systems, compositions, and methods to sort and sequence >10,000 (e.g., >100,000) rare cells in human brains of different age. Many important cell types in the aging brain are underrepresented in current atlases due to their rarity. In some embodiments, the present invention provides an efficient approach to target and sequence these important omitted cell types, thereby capturing other rare brain cell types, including pericytes, smooth muscle cells, microglia, and subtypes of vascular and leptomeningeal cells. These cells are often missed due to their rarity, even though they clearly exist in the human cortex^{1,2} and thus are glaring omissions in existing brain atlases. Generating detailed atlases of the brain relies on high-throughput single cell sequencing. However, due to the limited throughput of existing microfluidic technologies, sequencing enough cells to generate a complete atlas is impractical. Rare cell types that play crucial roles are often missed, yielding incomplete atlases. For example, in a recent comparative study of primary motor cortex, 5- to 15-fold lower sampling of non-neuronal cells impacted the detection of rare cell types¹. In some embodiments, the extreme throughput of PIP-PACS supports isolation of rare and fragile brain cells for sequencing, filling key gaps in brain atlases.

In some embodiments, the present invention provides systems, compositions, and methods to isolate and sequence >10,000 (e.g., >100,000) rare cells from the anterior cingulate cortex (ACC) of human brain using the PACS-PIPseq approach to isolate >10,000 (e.g., >100,000) nuclei comprising rare cell types from the human anterior cingulate cortex by targeting multiple nuclear RNA and protein biomarkers.

In some embodiments, the systems, compositions, and methods of the present invention enhance large scale CRISPR screens with perturb-seq to identify edits that produce a desired phenotype as identified by gene expression. Conventionally, such screens require sequencing of great numbers of edited cells, only a small portion of which comprise the phenotype of interest and are wasteful because all cells must be sequenced. Systems, compositions, and methods provided herein significantly increase the efficiency of a perturb-seq experiment by enriching and focusing on specific gene expression signatures for sequencing, and discarding all untargeted cells. In some embodiments, millions of edited cells are barcoded then the beads are sorted based on specific markers that are consistent with the targeted gene expression. The cells are then single-cell sequenced to obtain the expression profile spectrum and corresponding edits that participate in their generation, thereby significantly reducing the amount of sequencing required to identify edits of interest.

### EXPERIMENTAL EXAMPLES

### Example 1—Scalable PIPseq Emulsification

PIPseq uses hydrogel particles to ‘template’ monodispersed droplets. The polyacrylamide templates are manufactured with scalable membrane emulsification capable of generating liters of particles per hour.⁵² By including acrydite-modified DNA handles during manufacture, the beads may be labelled with barcode sequences using split pool methods that scale to hundreds of billions of unique sequences.⁵³ We have developed libraries capable to uniquely barcode 1M cells. Similar to published methods,^{19,20} mRNA is captured and reverse transcribed using barcoded oligo-dTs. Labeled beads may also be purchased from commercial vendors compatible with PIPseq.⁵⁴ A key step in the implementation of PIPseq to large scale experiments is emulsification of samples in well plates. Well plates afford an alternative means by which to scale single cell genomics, because they allow multiple samples to be processed simultaneously. In addition, when different samples are prepared on the same plate, coordinate hashing allows bioinformatic deconvolution after sequencing^{49,55}. With PIPseq, a single well on a 384 plate can accommodate >10,000 nuclei, allowing ˜4 million nuclei to be barcoded per plate. The total oligonucleotide and reagent cost for PIP barcoding at this scale (100 million PIPs) is ˜$1,000, with the majority being PCR enzyme and Nextera reagents (˜$800)⁵⁶, for a net cost of ˜0.01 cent/cell. This is substantially less than the current 10X Genomics cost per cell that arises for a variety of reasons, including: the substantial markup on their kits (˜90%), the requirement of a microfluidic platform and associated reagents⁴⁴, and the costly reverse transcription reaction performed in droplets rather than in bulk and thus using 100X the enzyme⁴⁴. For example, barcoding just 1 million cells using 10X Genomic's recently announced “high throughput” kit⁵⁷ costs ˜$200,000 in cell preparation alone.

### Example 2—Comparing PIPseq to 10X Genomics Chromium

In experiments conducted in development of the present invention, we constructed a single cell mRNA sequencing workflow that leverages the simplicity and scalability of PIP technology. The PIPseq workflow uses droplets to barcode the mRNA of cells. Instead of microfluidic encapsulation, the cells are mixed with the PIPs and oil, and vortexed for 1 min. Once encapsulated, thermal activation of proteinase K lyses the cells, resulting in mRNA release and hybridization to the bead. Fluorinated oil and PFPE-PEG surfactant stabilize the emulsion. The mixture is demulsified, and the particles recovered by centrifugation and washed. The mRNA is converted to cDNA by reverse transcription, amplified, adapters added by Nextera transposase, and sequenced¹⁹. In experiments conducted in development of the present invention, we observed that PIPseq provides excellent data with cells from myriad tissue types, including blood, breast, and brain. We further performed head-to-head comparisons with a conventional microfluidic approach (10X Chromium). We integrated PIPseq data across participants and recovered expected cell types by dimensionality reduction, including 2 lineages of luminal epithelial cells (LEP1 and LEP2), myoepithelial cells, fibroblasts, vascular cells, and immune cells. The comparative methods measured similar numbers of genes, transcripts, and cell type abundances, and clustering. Expected marker genes were concordant, with gene expression highly correlated (R²=0.99) and breast tissue markers from prior reports segregating identically within integrated clusters. This head-to-head comparison with the best-in-class single cell genomics platform (10X Chromium) demonstrates that PIPseq performs equivalently, but at a fraction of the cost and with much greater scalability. Therefore, PIPseq provides an enrichment-based technology for isolating and sequencing rare nuclei.

### Example 3—PIP Sorting with ddPCR Detection

An unmet need is to detect nuclei with specific mRNA biomarkers using a sensitive assay, then to isolate them for whole transcriptome sequencing. FISH is compatible with sorting but lacks the requisite sensitivity and interferes with transcriptome sequencing. Our strategy is to use ddPCR to detect targeted mRNA biomarkers. ddPCR has capability to detect single molecules. Because mRNA is stabilized as cDNA on PIPs during this step, the requisite thermal cycling does not degrade transcriptome data, supporting accurate downstream sequencing. In experiments conducted in development of the present invention, we demonstrated the ability to sensitively detect single mRNA transcripts and use this as a basis for sorting. We performed a dilution series of the target to detect positive droplets based on their fluorescence. The results were consistent with Poisson target loading typical of ddPCR and permitted accurate target quantitation over 4 decades. We next demonstrated this signal was sufficient for sorting the positive PIP droplets. Because the signal was a soluble TaqMan reporter, we used a droplet microfluidic sorter to sort the positives, that we had previously built. To select the positives, we gated according to fluorescence and observed proper sorting. While this demonstrated the feasibility of using ddPCR for PIP detection and sorting, the requirement of a custom microfluidic sorter is a major drawback that would impede adoption. To address this issue, we sought to remove the need for microfluidics by using instead a common flow cytometer. This, however, requires transferring the PIPs into a FACS-compatible aqueous phase while retaining the fluorescence signal. To accomplish this, we modified the PIPs with primers that captured the fluorescent ddPCR amplicons using a dual layered particle with polyacrylamide (PAA) core and agarose shell. Encapsulation happens in the surrounding aqueous phase. PCR cycling melts the agarose shell and fluorescence labeled primers are tethered onto the PAA. After thermocycling, the agarose solidifies and captures the genomic DNA. Upon recovering and washing the PIPs, unbound primers and amplicons are removed, revealing the positive PIPs. These particles at 50 μm in diameter and bright are easily sortable with a flow cytometer. The result is a microfluidic-free approach for barcoding, detecting, and isolating rare cells of interest.

### Example 4—Nuclear Isolation for snRNA-seq

With extensive experience handling brain tissue and preparing nuclei for single cell sequencing, we typically homogenize 500 mg of sectioned brain tissue in 5 mL of RNAase-free lysis buffer on ice using a glass dounce homogenizer, followed by sucrose density gradient centrifugation, and hemocytometer counting to yield >10M nuclei per 500 mg of tissue.

### Example 5—Enrichment of Rare Brain Cells Using Microfluidic PACS Sorting

To demonstrate the feasibility of sorting brain nuclei to isolate a specific rare cell type defined by a transcriptional biomarker, we used microfluidic PACS for the sorting, and performed pooled rather than single cell sequencing downstream. The goal was to isolate and sequence a rare astrocyte population from a mouse model of multiple sclerosis. IRE1α-XBP1 has been identified as a disease promoting pathway in specific astrocyte populations^{60,61}, but further characterizing these populations has remained difficult. IREla phosphorylation controls XBP1 splicing, removing a stop codon to produce the full-length transcription factor. XBP1 drives expression of downstream genes, including EDEM1, which is involved in the endoplasmic reticulum (ER) misfolded protein response. Using PACS, we sorted a pathogenic astrocyte subpopulation characterized by XBP1 signaling with the downstream target EDEMI as a marker of this subpopulation. We performed differential expression between astrocytes expressing EDEM1 and those not expressing EDEM1, performed upstream regulator analysis, and recovered XBP1 as a regulator of the transcriptional response. We applied PACS to sort and sequence a unique mouse astrocyte subpopulation and recovered the predicted upstream regulator that marked the sorted cells. This work demonstrates that PACS is a capable technology for isolating rare, understudied brain cells. In some embodiments, the technology is extended to nuclei with barcoded PIPs, thereby unlocking the ability to perform studies on postmortem human brain samples for detailed single cell data.

### Example 6—Microfluidics-Free PACS Nuclei Sequencing

In some embodiments, the present invention provides a microfluidic-free technique for sorting brain nuclei based on RNA biomarkers. There are 3 challenges for a microfluidics-free PACS nuclei sequencing method: 1) accurate detection of mRNA should be confirmed in PIPs on nuclei; 2) transcriptome information should be retained through detection; and 3) sorting should be transitioned from custom microfluidics to FACS, which is widely available. The PIPseq method, in contrast to 10x Genomics, retains barcodes on solid beads and is thus amenable to bead re-isolation by amplicon tethering and FACS. This approach is validated using 2-cell experiments to test sensitivity for detecting and sorting rare cells based on mRNA biomarkers using the full PIPs workflow with amplicon tethering. Human and mouse neuronal cell lines are cultured, with mixing experiments performed at serially diluted ratios of human:mouse (1:0, 1:1, 1:10, 1:100, 1:1000, 1:10000, and 0:1). Nuclei are purified, and 1000 nuclei are FACS sorted in triplicate, on a human-specific RNA biomarker (e.g., full length CDK5RAP2 varient^{62,63}). Barcoded PIPs are used for transcriptome capture, to support single cell deconvolution after sequencing of the sorted beads, and single nuclei used to test the accuracy of recovered transcriptomes recovered. Profiling the brain requires unique cell handling because the tissues cannot easily be disaggregated into intact single cell suspensions. Thus, it is now standard in brain atlasing efforts to sequence nuclei, which allow recovery of high-quality transcriptome, albeit with lower mRNA yields. As a result, efficient mRNA capture is an important part of developing nuclei-compatible scRNA-seq technologies. Our approach is superior to Drop-seq, which has demonstrated the ability to sequence nuclei. Because PIPseq is more sensitive than Drop-seq and applicable to nuclei, high data quality from human brains is obtained²¹. Mixing studies are performed because they clearly diagnose issues with cross-contamination and sort purity. 1:100 human: mouse nuclei are mixed, reverse transcription performed and ddPCR is used target neuronal mRNA markers. In parallel, PIPs that have not undergone a ddPCR mRNA detection step are used to quantify cDNA degradation. A PIPseq bioinformatics pipeline is used process single nuclei RNA-seq using pre-mRNA reference file (ENSEMBL GRCh38) to insure captured intronic reads originating from pre-mRNA transcripts are abundant in the nuclear fraction. In previous comparisons to 10X Genomics, this software performed equally to CellRanger software, and has the benefit of ensuring that datasets are processed identically. Individual expression matrices containing numbers of Unique molecular identifiers (UMIs) per nucleus per gene are filtered to retain nuclei with >500 genes and fewer than <5% of total UMIs originating from ribosomal RNAs. In some embodiments, high resolution differentiation during the sorting step is performed by multiplexing the assay. ddPCR is compatible with multiplexed amplification of thousands of targets at the single cell level, which is the core capability behind a technology we previously developed,^{23,64,65} Additionally, using combinations of fluorescence spectral and intensity encoding, up to 40 targets can be quantified optically, as in the commercially available multiplexed ddPCR instrument from Bio-Rad and Stilla Technologies⁶⁶⁻⁶⁹. Thus, it is possible to scale the number of markers to differentiate between related cell types during the sorting step of PIP-PACS. In some embodiments, additional colors are added to multiplex the reaction. Additional markers can be added with more colors and by implementing intensity encoding, although at the expense of complicating real-time analysis. Because each cell has a unique barcode, several potential technical concerns arise. Cross-hybridization with similar barcodes may occur, but specificity is ensured by single cell-sequencing. Unintended barcodes that cross-hybridize are easily identified by their sequence and removed bioinformatically. An alternative approach is to use ddPCR primers that only amplify specific barcodes (dial-out PCR)⁷⁰.

### Example 7—Transcriptomes of VENs Using PIP-PACS

De-identified snap-frozen post-mortem tissue samples from 4 neurotypical control donors, 2 male and 2 female, obtained from the NIH NeuroBioBank are used for PIP-PACS. The ACC is dissected, and cortical samples encompassing the entire span of the cortex are coronally cryosectioned at 100 μm, dissociated, and nuclei extracted as in Aim 1. Total RNA from ˜10 mg of collected tissue is isolated and used to perform RNA integrity analysis on the Agilent 2100 Bioanalyzer using the RNA Pico Chip assay. Only samples with RNA integrity number (RIN) >6.5 are used to perform nuclei isolation. Sorting VENs: GABRQ and ITGA4 are both genes highly enriched in VENs, but also expressed in Layer 5 fork cells.⁷ The localization and specificity of these genes for VENs is confirmed by RNA in situ hybridization in the ACC tissue sections using RNAScope. GABRQ is used as a mRNA label to enrich VENs for PIP-PACS. The nuclei collected from ACC samples are sorted using 2 markers, the neuron-specific nuclear marker NEUN to eliminate non-neural cells⁷¹, and GABRQ to enrich for VENs. NEUN+/GABRQ+ nuclei enriched for VENs are sorted and collected, and NEUN+/GABRQ-nuclei depleted of VENs for comparison of gene expression from Von Economo and fork neurons, with other neurons from ACC. ˜10,000 VEN nuclei are collected. There are ˜5 morphologically identified VENs per 1,000 pyramidal neurons in regions containing VENs72, so ˜2 million pyramidal cell nuclei are required recovered from ˜200 mg of tissue⁵. However, to accommodate for wastage and provide a margin for elimination of poor quality nuclei, sorting begins with >500 mg of tissue. Deep sequencing VEN data is compared to previously recovered low coverage data, allowing comparison of results and confirming biological conclusions. This dataset is of value for comparative studies that shed additional light on the evolution of VENs which have only been observed in a limited number of species. These data provide a basis for identifying VEN cell types derived from human stem cells, a major advance for disease modeling^{73.74}. Iterative clustering is performed to group nuclei by gene expression similarity. Clusters containing cells from a single donor and nuclei mapping to low-quality outlier clusters are excluded. Our deep sampling method provides sufficient nuclei to subcluster the VEN population and identify a transcriptomic cell type signature for VEN cells and related Layer 5 neurons such as the closely-related fork cells.⁷ Genes differentially enriched in specific cell types are validated by subcellular spatial expression visualized by in situ hybridization in tissue sections by RNAScope. Immunolabelled ACC sections are used to characterize cell morphology and spatial distribution across cortical layers and regions. Differential expression analysis is performed to compare VEN clusters to other excitatory neurons to identify sets of genes selectively expressed in VENs. Results are compared to existing VEN gene expression datasets, including from non-human species^{7,75} to determine how human VENs compare to related cell types across species. >500 mg of tissue is obtained from ACC from each sample, similar to that we have obtained from samples previously⁷⁶ to yield >10M nuclei. Additional samples are pooled to reach this number if nuclei counts are lower. In some embodiments, additional genes are added as the PACS method can be multiplexed^{45.77}, including ITGA4 known to be enriched in VENs⁷. A single specific VEN marker gene has not been identified and may not exist, although gene combinations are known to identify this cell type and are targetable with PACS. In addition, the throughput of PACS yields VENs in much larger numbers than have been achieved to date, which supports deep exploration of their gene expression and signaling pathways yielding antibody markers of use to enrich these cells. In some embodiments, this approach is applied neonatal age anterior cingulate cortex samples for comparison because VENs are the only known neurons to increase in number postnatally, between birth and four years.⁶ Changes in cell types or states and differential gene expression compared to the adult VEN data highlights developmental changes with disease significance^{3,6}.

### Example 8—Sorting and Sequencing Low Abundance Brain Cells

In some embodiments, using a multiplexed assay the present invention supports sorting of extremely rare brain nuclei using a multiplexed assay. Human brain extracted nuclei are encapsulated and lysed, followed by capturing and barcoding their mRNA on PIP beads. The beads are subjected to multiplexed, fluorescence amplicon tethering ddPCR to identify cells of interest according to characteristic biomarkers, including GABAergic interneurons, VENs, Cajal-Retzius cells, and Layer 6b projection neurons (e.g., NeuN, GABRQ, GAD67, CALB2, CTGF, NR4A2),^{71,78-81}, additional markers are used for blood vessels,⁸² pericytes⁸³, microglia⁸⁴, and adult neural progenitors^{85,86}. Using model mixtures of human and mouse brain extracted nuclei, accurate and sensitive rare nuclei sorting and sequencing is provided. To test the efficiency with which PIP-PAC isolate extremely rare cells from spike-in samples using mouse and human whole cell lines, we generated mixtures from 1:10 to 1:100,000 rarity and, in all cases, obtained pure single cell data with high read counts. In addition, we've demonstrated isolation of cells with a rarity of 1 in a million, sorting 30 million total cells in 30 min to recover 10 target cells. In these experiments, we used “yield mode” on the flow cytometer, accepting a high frequency of false positives to prioritize capture of the extremely rare cells, since all sorted cells are individually barcoded and sequenced, and falsely sorted cells can be identified and excluded bioinformatically. In some embodiments, nucleic rather than cells are used with probes associated with the rare brain cells of interest, as cited above. To streamline processing of different brains and regions, well plate PIP-PACS is implements using the native capability of a flow cytometer to dispense into 96 well plates. Each nuclei sample from different individuals and brain regions is processed with PIP barcoding, then sorted with the flow cytometer, pooling each samples positive nuclei into a defined well on the collection plate. The barcoded nuclei for each sample is then indexed, allowing them to be pooled and sequenced, greatly streamlining multi-sample processing. In some embodiments, well plate experiments with different plate and shaker geometries establish the simplest large-format emulsification conditions possible. To screen a large condition space (e.g., plate size, vortexer speed and geometry, time, bead-to-oil ratio), emulsion quality and shell thickness is assessed with microscopy to quickly determine the robustness of emulsification to these parameters, and provide practical rules for large format studies. The American Type Culture Collection (ATCC) provides commercial access to human, mouse (Mus musculus), rat (Rattus norvegicus), cat (Felis catus), quail (Coturnix coturnix japonica), and monkey (Macaca mulatta) nervous system cell lines⁸⁷. In some embodiments, we use the human, mouse, and monkey nuclei, which allows us to quickly assess cross-contamination, emulsion quality, and cell type bias. Samples are run from the outer corner wells, the midline wells, and the innermost center wells. In all cases, samples from each well are individually sequenced and analyzed to determine variance across the plate. As a control, we use the standard format for the same number of nuclei. As well, we use the cultured cell lines to estimate sensitivity and purity of the data. Human, mouse, and monkey nuclei are tested at 1:1:1 and 4:2:1 ratios, respectively, to assess capture bias observed in single cell studies^{88,89}. Sequencing data are analyzed for diagnostic quality markers and cell type purity within a given barcode group, comparison of the bioinformatic cell type abundance relative to experimental input, number of detected UMIs, number of reads and gene calls per cell, and quality read lengths per cell type⁹⁰⁻⁹². To ensure consistency in data handling, we use the scumi workflow developed for comparing scRNA-seq methods and these rubrics⁴³. Based on these parameters, we tune the process to optimize overall data and nuclei capture efficiency. To support detection of cell type specific nuclear transcription factor proteins, it is important to fix and permeabilize nuclei⁹³ with a process that does not interfere with downstream barcoding and RNA sequencing. Harsh fixation can fragment nucleic acids or generate adducts that do not effectively barcode, leading to drop out⁹⁴⁻⁹⁶. Additionally, certain chemistries such as pure methanol denature proteins and preclude antibody staining⁹⁷. Towards this end, we adapt components of the protocols for fixation and handling that have recently been reported in methods like INs-Seq98, Probe-Seq33, and Flow-FISH99. Fixation protocols, staining and hybridization methods, and incubation times are optimized as needed. This includes different fixation methods including low concentration methanol and dithio-bis(succinimidyl propionate) (DSP)100, or reversible fixatives, such as Glyoxal, 101 that further improve downstream molecular assays.

### Example 9—Isolations and Sequencing of >100,000 Rare Cells from the Anterior Cingulate Cortex (ACC)

To isolate and sequence >100,00 rare cells from the human ACC, de-identified snap-frozen tissues from the left hemisphere are obtained from the NIH NeuroBioBank¹⁰². The cingulate gyrus, taken posterior to the optic chiasm between the infundibulum and mammillary body is removed and cortical samples encompassing the entire span of the cortex is vibratome sectioned. Total RNA from ˜10 mg of collected tissue is isolated and used to perform RNA integrity analysis on the Agilent 2100 Bioanalyzer using RNA Pico Chip assay. Only samples with RNA integrity number (RIN)103 >6.5 are used for single-nucleus RNA sequencing (snRNA-seq). In experiments conducted in development of the present invention, we used unbiased, high throughput single nucleus sequencing to detect these cells. Because no enrichment was performed, transcriptome coverage and sequencing were limited, although the results clearly demonstrate that these cells are captured with the method. The results also confirm the RNA marker panel used for sorting. By implementing enrichment, coverage of these rare cells is increased by over 100X, to significantly increase cell type clustering and transcriptome quality to fill out the atlas. Beyond targeting neurons, we multiplex the capture of pericytes, smooth muscle cells, microglia, and subtypes of vascular and leptomeningeal cells not typically represented in brain atlases, even though they clearly exist in the human cortex^{1,2}. We target these cells by sorting for expression of distinctive markers for neurons of known rarity like GABAergic interneurons, VENs, Cajal-Retzius cells, and Layer 6b projection neurons (e.g., NeuN, GABRQ, GAD67, CALB2, CTGF, NR4A2).^{71,78-81} Additional markers are used for blood vessels (CD31),⁸² pericytes (CD13)⁸³, microglia (CD11b, CD45)⁸⁴, and adult neural progenitors (SOX2, CD133)^{85,86}. Barcoded nuclei are sorted into wells according to sample type in triplicate and processed for library preparation according to the optimized PIPseq protocol. Libraries are quantified via bioanalyzer to confirm RNA integrity and yield before processing to adaptor ligation and sequencing. Sequence data are subjected to principal component analysis (PCA) dimensionality reduction using Seurat V4 selecting components based on a scree plot, followed by Louvain clustering and Uniform Manifold Approximation and Projection (UMAP)¹⁰⁴⁻¹⁰⁶. Cell types are annotated based on expression of marker genes and visualized on the UMAP plot using Seurat's FeaturePlot function and by performing unbiased gene marker analysis using the FindMarkers function. Use of mRNA FISH probes to label and sort nuclei based on the expression of cell types with known cluster of differentiation (CD) markers achieved low sensitivity and recovered few cells. The ddPCR-based detection of PIP-PACS of the present invention, being an exponential reaction, is yields much greater sensitivity to detect these often-minimally expressed markers, and thus higher efficiency for recovering these rare cells. PIP-PACS is also compatible with antibody staining of nuclear proteins, which may be preferable to mRNAs for certain cell types. In the approach that we previously developed, published^{107,108}, and made commercially available through Bio-Legend, Mission Bio, and 10X genomics^{23,65,109,110}, oligo-conjugated antibodies are used to stain nuclear proteins, thereby physically linking them to a nucleic acid suitable for ddPCR detection and single nucleus barcoding (CITE-seq)^{48,111}. Because PIP-PACS uses proteinase K to digest cells, it is compatible with the requisite fixation and antibody staining steps, allowing capture of nuclear mRNAs and antibody tags on the PIP bead for ddPCR detection and barcoding. Compared to direct detection of bound antibody probes by fluorescence, ddPCR detection of antibody oligo tags is more sensitive because the assay is exponential, yielding a bright, sortable signal. Using our synthetic cell mixtures, we test for enrichment of target populations and access the quality of the resulting data using the accepted rubrics of knee plots, read counts, read quality, and read purity^{43,112}. PIP-PACS is also compatible with pre-tagmentation of nuclei to perform single nuclei ATAC-seq that can be multiplexed with RNA-seq and Ab-seq/CITE-seq, like the microfluidic methods^{113,114}. In some embodiments, the present invention provides “multiomic” characterization to significantly enhance data quality and cell type clustering.

### REFERENCES

1. Bakken T E, Jorstad N L, Hu Q, Lake B B, Tian W, Kalmbach B E, Crow M, Hodge R D, Krienen F M, Sorensen S A, Eggermont J, Yao Z, Aevermann B D, Aldridge A I, Bartlett A, Bertagnolli D, Casper T, Castanon R G, Crichton K, Daigle T L, Dalley R, Dee N, Dembrow N, Diep D, Ding S L, Dong W, Fang R, Fischer S, Goldman M, Goldy J, Graybuck L T, Herb B R, Hou X, Kancherla J, Kroll M, Lathia K, Lew B van, Li Y E, Liu C S, Liu H, Mahurkar A, McMillen D, Miller J A, Moussa M, Nery J R, Orvis J, Owen S, Palmer C R, Pham T, Plongthongkum N, Poirion O, Reed N M, Rimorin C, Rivkin A, Romanow W J, Sedeño-Cortés A E, Siletti K, Somasundaram S, Sulc J, Tieu M, Torkelson A, Tung H, Wang X, Xie F, Yanny A M, Zhang R, Ament S A, Bravo H C, Chun J, Dobin A, Gillis J, Hertzano R, Hof P R, Höllt T, Horwitz G D, Keene C D, Kharchenko P V, Ko A L, Lelieveldt B P, Luo C, Mukamel E A, Preissl S, Regev A, Ren B, Scheuermann R H, Smith K, Spain W J, White O R, Koch C, Hawrylycz M, Tasic B, Macosko E Z, McCarroll S A, Ting J T, Zeng H, Zhang K, Feng G, Ecker J R, Linnarsson S, Lein E S. Evolution of cellular diversity in primary motor cortex of human, marmoset monkey, and mouse. bioRxiv. Cold Spring Harbor Laboratory; 2020;
2. Thrupp N, Sala Frigerio C, Wolfs L, Skene N G, Fattorelli N, Poovathingal S, Fourne Y, Matthews P M, Theys T, Mancuso R, de Strooper B, Fiers M. Single-Nucleus RNA-Seq Is Not Suitable for Detection of Microglial Activation Genes in Humans. Cell Reports. 2020; 32(13):108189.
3. Allman J M, Watson K K, Tetreault N A, Hakeem A Y. Intuition and autism: a possible role for Von Economo neurons. Trends Cogn Sci. 2005; 9(8):367-373. PMID: 16002323
4. Smith G E. Die Cytoarchitektonik der Hirnrinde des erwachsenen Menschen. J Anat. 1927; 61(Pt 2):264-266. PMCID: PMC 1249949
5. Nimchinsky E A, Gilissen E, Allman J M, Perl D P, Erwin J M, Hof P R. A neuronal morphologic type unique to humans and great apes. Proceedings of the National Academy of Sciences. 1999; 96(9):5268-5273.
6. Weickert C S, Webster M J, Colvin S M, Herman M M, Hyde T M, Weinberger D R, Kleinman J E. Localization of epidermal growth factor receptors and putative neuroblasts in human subependymal zone. J Comp Neurol. 2000; 423(3):359-372.
7. Hodge R D, Miller J A, Novotny M, Kalmbach B E, Ting J T, Bakken T E, Aevermann B D, Barkan E R, Berkowitz-Cerasano M L, Cobbs C, Diez-Fuertes F, Ding S L, McCorrison J, Schork N J, Shehata S I, Smith K A, Sunkin S M, Tran D N, Venepally P, Yanny A M, Steemers F J, Phillips J W, Bernard A, Koch C, Lasken R S, Scheuermann R H, Lein E S. Transcriptomic evidence that von Economo neurons are regionally specialized extratelencephalic-projecting excitatory neurons. Nature Communications. Nature Publishing Group; 2020; 11(1):1172.
8. Lein E S, Hawrylycz M J, Ao N, Ayres M, Bensinger A, Bernard A, Boe A F, Boguski M S, Brockway K S, Byrnes E J, Chen L, Chen L, Chen T M, Chi Chin M, Chong J, Crook B E, Czaplinska A, Dang C N, Datta S, Dee N R, Desaki A L, Desta T, Diep E, Dolbeare T A, Donelan M J, Dong H W, Dougherty J G, Duncan B J, Ebbert A J, Eichele G, Estin L K, Faber C, Facer B A, Fields R, Fischer S R, Fliss T P, Frensley C, Gates S N, Glattfelder K J, Halverson K R, Hart M R, Hohmann J G, Howell M P, Jeung D P, Johnson R A, Karr P T, Kawal R, Kidney J M, Knapik R H, Kuan C L, Lake J H, Laramee A R, Larsen K D, Lau C, Lemon T A, Liang A J, Liu Y, Luong L T, Michaels J, Morgan J J, Morgan R J, Mortrud M T, Mosqueda N F, Ng L L, Ng R, Orta G J, Overly C C, Pak T H, Parry S E, Pathak S D, Pearson O C, Puchalski R B, Riley Z L, Rockett H R, Rowland S A, Royall J J, Ruiz M J, Sarno N R, Schaffnit K, Shapovalova N V, Sivisay T, Slaughterbeck C R, Smith S C, Smith K A, Smith B I, Sodt A J, Stewart N N, Stumpf K R, Sunkin S M, Sutram M, Tam A, Teemer C D, Thaller C, Thompson C L, Varnam L R, Visel A, Whitlock R M, Wohnoutka P E, Wolkey C K, Wong V Y, Wood M, Yaylaoglu M B, Young R C, Youngstrom B L, Feng Yuan X, Zhang B, Zwingman T A, Jones A R. Genome-wide atlas of gene expression in the adult mouse brain. Nature. Nature Publishing Group; 2007; 445(7124):168-176.
9. Hawrylycz M J, Lein E S, Guillozet-Bongaarts A L, Shen E H, Ng L, Miller J A, van de Lagemaat L N, Smith K A, Ebbert A, Riley Z L, Abajian C, Beckmann C F, Bernard A, Bertagnolli D, Boe A F, Cartagena P M, Chakravarty M M, Chapin M, Chong J, Dalley R A, Daly B D, Dang C, Datta S, Dee N, Dolbeare T A, Faber V, Feng D, Fowler D R, Goldy J, Gregor B W, Haradon Z, Haynor D R, Hohmann J G, Horvath S, Howard R E, Jeromin A, Jochim J M, Kinnunen M, Lau C, Lazarz E T, Lee C, Lemon T A, Li L, Li Y, Morris J A, Overly C C, Parker P D, Parry S E, Reding M, Royall J J, Schulkin J, Sequeira P A, Slaughterbeck C R, Smith S C, Sodt A J, Sunkin S M, Swanson B E, Vawter M P, Williams D, Wohnoutka P, Zielke H R, Geschwind D H, Hof P R, Smith S M, Koch C, Grant S G N, Jones A R. An anatomically comprehensive atlas of the adult human brain transcriptome. Nature. Nature Publishing Group; 2012; 489(7416):391-399
10. Ero C, Gewaltig M O, Keller D, Markram H. A Cell Atlas for the Mouse Brain. Front Neuroinform. Frontiers; 2018; 12:84.
11. Herculano-Houzel S, Mota B, Lent R. Cellular scaling rules for rodent brains. Proc Natl Acad Sci U S A. 2006; 103(32):12138-12143. PMCID: PMC 1567708
12. von Bartheld C S. Myths and truths about the cellular composition of the human brain: A review of influential concepts. Journal of Chemical Neuroanatomy. 2018; 93:2-15.
13. Falconer D S, Gauld I K, Roberts R C. Cell numbers and cell sizes in organs of mice selected for large and small body size. Genet Res. 1978; 31(3):287-301. PMID: 689373
14. Bianconi E, Piovesan A, Facchin F, Beraudi A, Casadei R, Frabetti F, Vitale L, Pelleri M C, Tassani S, Piva F, Perez-Amodio S, Strippoli P, Canaider S. An estimation of the number of cells in the human body. Ann Hum Biol. 2013; 40(6):463-471. PMID: 23829164
15. Wilbrey-Clark A, Roberts K, Teichmann S A. Cell Atlas technologies and insights into tissue architecture. Biochem J. 2020; 477(8):1427-1442. PMCID: PMC 7200628
16. Taylor D M, Aronow B J, Tan K, Bernt K, Salomonis N, Greene C S, Frolova A, Henrickson S E, Wells A, Pei L, Jaiswal J K, Whitsett J, Hamilton K E, MacParland S A, Kelsen J, Heuckeroth R O, Potter S S, Vella L A, Terry N A, Ghanem L R, Kennedy B C, Helbig I, Sullivan K E, Castelo-Soccio L, Kreigstein A, Herse F, Nawijn M C, Koppelman G H, Haendel M, Harris N L, Rokita J L, Zhang Y, Regev A, Rozenblatt-Rosen O, Rood J E, Tickle T L, Vento-Tormo R, Alimohamed S, Lek M, Mar J C, Loomes K M, Barrett D M, Uapinyoying P, Beggs A H, Agrawal P B, Chen Y W, Muir A B, Garmire L X, Snapper S B, Nazarian J, Seeholzer S H, Fazelinia H, Singh L N, Faryabi R B, Raman P, Dawany N, Xie H M, Devkota B, Diskin S J, Anderson S A, Rappaport E F, Peranteau W, Wikenheiser-Brokamp K A, Teichmann S, Wallace D, Peng T, Ding Y yang, Kim M S, Xing Y, Kong S W, Bönnemann C G, Mandl K D, White P S. The Pediatric Cell Atlas: Defining the Growth Phase of Human Development at Single-Cell Resolution. Developmental Cell. 2019; 49(1):10-29.
17. Boehm J S, Garnett M J, Adams D J, Francies H E, Golub T R, Hahn W C, Iorio F, McFarland J M, Parts L, Vazquez F. Cancer research needs a better map. Nature. 2021; 589(7843):514-516.
18. Hon C C, Shin J W, Carninci P, Stubbington M J T. The Human Cell Atlas: Technical approaches and challenges. Briefings in Functional Genomics. 2018; 17(4):283-294.
19. Macosko E Z, Basu A, Satija R, Nemesh J, Shekhar K, Goldman M, Tirosh I, Bialas A R, Kamitaki N, Martersteck E M, Trombetta J J, Weitz D A, Sanes J R, Shalek A K, Regev A, McCarroll S A. Highly Parallel Genome-wide Expression Profiling of Individual Cells Using Nanoliter Droplets. Cell. 2015; 161(5):1202-1214. PMCID: PMC 4481139
20. Klein A M, Mazutis L, Akartuna I, Tallapragada N, Veres A, Li V, Peshkin L, Weitz D A, Kirschner M W. Droplet Barcoding for Single-Cell Transcriptomics Applied to Embryonic Stem Cells. Cell. 2015; 161(5):1187-1201.
21. Habib N, Avraham-Davidi I, Basu A, Burks T, Shekhar K, Hofree M, Choudhury S R, Aguet F, Gelfand E, Ardlie K, Weitz D A, Rozenblatt-Rosen O, Zhang F, Regev A. Massively parallel single-nucleus RNA-seq with DroNc-seq. Nat Methods. 2017; 14(10):955-958.
22. 10X Genomics. Chromium Genome Solution [Internet]. 2017 [cited 2018 Oct. 30]. Available from: http://go.10xgenomics.com/1/172142/2016-08-10/3svk9/172142/8086/LIT00003_RevB_Chromium_Genome_Solution_Application_Note_Digi tal.pdf
23. Mission Bio, Inc. Mission Bio Tapestri Platform [Internet]. 2018. Available from: https://missionbio.com/content/uploads/2018/03/Mission-Bio_Product-Brochure_march_2018_final_web_ready.pdf.
24. Svensson V, Vento-Tormo R, Teichmann S A. Exponential scaling of single-cell RNA-seq in the past decade. Nature Protocols. 2018; 13(4):599-604.
25. Liu J, Hansen C, Quake S R. Solving the “World-to-Chip” Interface Problem with a Microfluidic Matrix. Anal Chem. American Chemical Society; 2003; 75(18):4718-4723.
26. 10X Genomics. Chromium Single Cell 3′Reagent Kits v3 [Internet]. 2020 [cited 2021 Mar. 3]. Available from: https://support.10xgenomics.com/single-cell-gene-expression/library-prep/doc/user-guide-chromium-single-cell-3-reagent-kits-user-guide-v3-chemistry
27. Illumina, Inc. NovaSeq 6000 Sequencing System [Internet]. 2020. Available from: https://emea.illumina.com/content/dam/illumina/gcs/assembled-assets/marketing-literature/novaseq-6000-spec-sheet-770-2016-025/novaseq-6000-spec-sheet-770-2016-025.pdf
28. NCI Office of Science and Technology Resources. Sequencing Facility—OSTR [Internet]. [cited 2021 Mar. 2]. Available from: https://ostr.ccr.cancer.gov/resources/sequencing-facility/
29. Element Biosciences Unveils New DNA Sequencing Instrument, Chemistry [Internet]. Genomeweb. 2022 [cited 2022 Jul. 24]. Available from: https://www.genomeweb.com/sequencing/element-biosciences-unveils-new-dna-sequencing-instrument-chemistry
30. Pennisi E. Upstart DNA sequencers could be a ‘game changer.’ Science. 2022; 376(6599):1257-1258.
31. Hatori M N, Kim S C, Abate A R. Particle-Templated Emulsification for Microfluidics-Free Digital Biology. Anal Chem. 2018; 90(16):9813-9820. PMCID: PMC6122844
32. Mattei D, Ivanov A, van Oostrum M, Pantelyushin S, Richetto J, Mueller F, Beffinger M, Schellhammer L, vom Berg J, Wollscheid B, Beule D, Paolicelli RC, Meyer U. Enzymatic Dissociation Induces Transcriptional and Proteotype Bias in Brain Cell Populations. IJMS. 2020; 21(21):7944.
33. Amamoto R, Garcia M D, West E R, Choi J, Lapan S W, Lane E A, Perrimon N, Cepko C L. Probe-Seq enables transcriptional profiling of specific cell types from heterogeneous tissue by RNA-based isolation. Guillemot F, Bronner M E, Shaffer S, editors. eLife. eLife Sciences Publications, Ltd; 2019; 8:e51452.
34. Eastburn D J, Sciambi A, Abate A R. Picoinjection Enables Digital Detection of RNA with Droplet RT-PCR. Chin W C, editor. PLoS ONE. 2013; 8(4):e62961. PMCID: PMC3637249
35. Eastburn D J, Sciambi A, Abate A R. Ultrahigh-throughput Mammalian single-cell reverse-transcriptase polymerase chain reaction in microfluidic drops. Anal Chem. 2013; 85(16):8016-8021. PMID: 23885761
36. Ramsköld D, Luo S, Wang Y C, Li R, Deng Q, Faridani O R, Daniels G A, Khrebtukova I, Loring J F, Laurent L C, Schroth G P, Sandberg R. Full-length mRNA-Seq from single-cell levels of RNA and individual circulating tumor cells. Nature Biotechnology. Nature Publishing Group; 2012; 30(8):777-782.
37. Jaitin D A, Kenigsberg E, Keren-Shaul H, Elefant N, Paul F, Zaretsky I, Mildner A, Cohen N, Jung S, Tanay A, Amit I. Massively Parallel Single-Cell RNA-Seq for Marker-Free Decomposition of Tissues into Cell Types. 2014; 343:5.
38. Gierahn T M, Wadsworth M H, Hughes T K, Bryson B D, Butler A, Satija R, Fortune S, Love J C, Shalek A K. Seq-Well: portable, low-cost RNA sequencing of single cells at high throughput. Nat Methods. 2017; 14(4):395-398.
39. Vitak S A, Torkenczy K A, Rosenkrantz J L, Fields A J, Christiansen L, Wong M H, Carbone L, Steemers F J, Adey A. Sequencing thousands of single-cell genomes with combinatorial indexing. Nat Methods. 2017; 14(3):302-308.
40. Honeycomb Biotechnologies, Inc. Multiomic Cellular Solutions—Any Cell. Anywhere. Anytime. [Internet]. [cited 2021 Mar. 1]. Available from: https://honeycomb.bio/
41. Parse Biosciences. Scalable Single Cell Sequencing [Internet]. [cited 2021 Mar. 1]. Available from: https://www.parsebiosciences.com
42. Cao J, Spielmann M, Qiu X, Huang X, Ibrahim D M, Hill A J, Zhang F, Mundlos S, Christiansen L, Steemers F J, Trapnell C, Shendure J. The single-cell transcriptional landscape of mammalian organogenesis. Nature. 2019; 566(7745):496-502.
43. Ding J, Xian Adiconis, Sean Simmons, Monika Kowalczyk, Cynthia Hession, Nemanja Marjanovic, Travis Hughes, Marc Wadsworth, Tyler Burks, Lan Nguyen, John Kwon, Boaz Barak, William Ge, Amanda Kedaigle, Shaina Carroll, Shuqiang Li, Nir Hacohen, Orit Rozenblatt-Rosen, Alex Shalek, Alexandra-Chloe Villani, Aviv Regev, Joshua Z. Levin. Systematic comparison of single-cell and single-nucleus RNA-sequencing methods. Nature Biotechnol. 2020; 38:26.
44. Zheng G X Y, Terry J M, Belgrader P, Ryvkin P, Bent Z W, Wilson R, Ziraldo S B, Wheeler T D, McDermott G P, Zhu J, Gregory M T, Shuga J, Montesclaros L, Underwood J G, Masquelier D A, Nishimura S Y, Schnall-Levin M, Wyatt P W, Hindson C M, Bharadwaj R, Wong A, Ness K D, Beppu L W, Deeg H J, McFarland C, Loeb K R, Valente W J, Ericson N G, Stevens E A, Radich J P, Mikkelsen T S, Hindson B J, Bielas J H. Massively parallel digital transcriptional profiling of single cells. Nat Commun. 2017; 8(1):14049.
45. Clark I C, Abate A R. Finding a helix in a haystack: nucleic acid cytometry with droplet microfluidics. Lab Chip. 2017; 17(12):2032-2045. PMCID: PMC 6005652
46. Clausell-Tormos J, Lieber D, Baret J C, El-Harrak A, Miller O J, Frenz L, Blouwolff J, Humphry K J, Köster S, Duan H, Holtze C, Weitz D A, Griffiths A D, Merten C A. Droplet-Based Microfluidic Platforms for the Encapsulation and Screening of Mammalian Cells and Multicellular Organisms. Chemistry & Biology. 2008; 15(5):427-437.
47. 10X Genomics. Our 1.3 million single cell dataset is ready to download [Internet]. [cited 2021 Mar. 1]. Available from: https://www.10xgenomics.com/blog/our-13-million-single-cell-dataset-is-ready-to-download
48. Stoeckius M, Zheng S, Houck-Loomis B, Hao S, Yeung B Z, Mauck W M, Smibert P, Satija R. Cell Hashing with barcoded antibodies enables multiplexing and doublet detection for single cell genomics. Genome Biol. 2018 Dec. 19; 19(1):224. PMCID: PMC 6300015
49. McGinnis C S, Patterson D M, Winkler J, Conrad D N, Hein M Y, Srivastava V, Hu J L, Murrow L M, Weissman J S, Werb Z, Chow E D, Gartner Z J. MULTI-seq: sample multiplexing for single-cell RNA sequencing using lipid-tagged indices. Nat Methods. 2019; 16(7):619-626. PMCID: PMC 6837808
50. Illumina, Inc. Innovation at Illumina: The road to the $600 human genome. Nature Portfolio [Internet]. 2021 [cited 2021 Mar. 2]; Available from: https://www.nature.com/articles/d42473-021-00030-9
51. Delley C L, Abate A R. Modular barcode beads for microfluidic single cell genomics. Sci Rep. 2021 May 25; 11(1):10857. PMCID: PMC 8149635
52. Kobayashi I, Neves M A, Wada Y, Uemura K, Nakajima M. Large microchannel emulsification device for mass producing uniformly sized droplets on a liter per hour scale. Green Processing and Synthesis. De Gruyter; 2012; 1(4):353-362.
53. Delley C L, Abate A R. Modular barcode beads for microfluidic single cell genomics. bioRxiv. Cold Spring Harbor Laboratory; 2020;
54. Dolomite Bio. scRNA-Seq Reagent Kit [Internet]. [cited 2021 Mar. 1]. Available from: https://www.dolomite-bio.com/product/reagent-kit/
55. Zhang J Q, Siltanen C A, Liu L, Chang K C, Gartner Z J, Abate A R. Linked optical and gene expression profiling of single cells at high-throughput. Genome Biol. 2020; 21(1):49. PMCID: PMC7041248
56. Li H, Wu K, Ruan C, Pan J, Wang Y, Long H. Cost-reduction strategies in massive genomics experiments. Mar Life Sci Technol. 2019; 1(1):15-21.
57. 10X Genomics. Xperience 2021 Event [Internet]. [cited 2021 Mar. 3]. Available from: https://pages.10xgenomics.com/xperience-resources.html
58. Eastburn D J, Sciambi A, Abate A R. Identification and genetic analysis of cancer cells with PCR-activated cell sorting. Nucleic Acids Res. 2014; 42(16):e128. PMCID: PMC4176366
59. Hatori M N, Modavi C, Xu P, Weisgerber D, Abate A R. Dual-layered hydrogels allow complete genome recovery with nucleic acid cytometry. Biotechnol J. 2022; 17(4):e2100483. PMID: 35088927
60. Wheeler M A, Clark I C, Tjon E C, Li Z, Zandee S E J, Couturier C P, Watson B R, Scalisi G, Alkwai S, Rothhammer V, Rotem A, Heyman J A, Thaploo S, Sanmarco L M, Ragoussis J, Weitz D A, Petrecca K, Moffitt J R, Becher B, Antel J P, Prat A, Quintana F J. MAFG-driven astrocytes promote CNS inflammation. Nature. 2020; 578(7796):593-599. PMCID: PMC8049843
61. Wheeler M A, Jaronen M, Covacu R, Zandee S E J, Scalisi G, Rothhammer V, Tjon E C, Chao C C, Kenison J E, Blain M, Rao V T S, Hewson P, Barroso A, Gutiérrez-Vázquez C, Prat A, Antel J P, Hauser R, Quintana F J. Environmental Control of Astrocyte Pathogenic Activities in CNS Inflammation. Cell. 2019; 176(3):581-596.e18. PMCID: PMC 6440749
62. Bitar M, Kuiper S, O'Brien E A, Barry G. Genes with human-specific features are primarily involved with brain, immune and metabolic evolution. BMC Bioinformatics. 2019 Nov. 22; 20(Suppl 9):406. PMCID: PMC 6873653
63. Park J S Y, Lee M K, Kang S, Jin Y, Fu S, Rosales J L, Lee K Y. Species-Specific Expression of Full-Length and Alternatively Spliced Variant Forms of CDK5RAP2. PLoS One. 2015; 10(11):e0142577. PMCID: PMC 4638350
64. Mission Bio, Inc. Mission Bio Co-Founder Awarded Grant from Chan-Zuckerberg Foundation [Internet]. Mission Bio. Available from: https://missionbio.com/press/mission-bio-co-founder-awarded-grant-from-chan-zuckerberg-foundation-renowned-single-cell-genomics-researcher-receives-funding-to-further-his-work/
65. Pellegrino M, Sciambi A, Treusch S, Durruthy-Durruthy R, Gokhale K, Jacob J, Chen T X, Geis J A, Oldham W, Matthews J, Kantarjian H, Futreal P A, Patel K, Jones K W, Takahashi K, Eastburn D J. High-throughput single-cell DNA sequencing of acute myeloid leukemia tumors with droplet microfluidics. Genome Res. 2018; 28(9):1345-1352.
66. Stilla. Guidelines for 6-color multiplex assay design for optimized performance with Crystal Digital PCRTM [Internet]. Stilla Technologies. Available from: https://www.stillatechnologies.com/guidelines-for-6-color-multiplex-assay-design-for-optimized-performance-with-crystal-digital-pcr/
67. Hindson B J, Ness K D, Masquelier D A, Belgrader P, Heredia N J, Makarewicz A J, Bright I J, Lucero M Y, Hiddessen A L, Legler T C, Kitano T K, Hodel M R, Petersen J F, Wyatt P W, Steenblock E R, Shah P H, Bousse L J, Troup C B, Mellen J C, Wittmann D K, Erndt N G, Cauley T H, Koehler R T, So A P, Dube S, Rose K A, Montesclaros L, Wang S, Stumbo D P, Hodges S P, Romine S, Milanovich F P, White H E, Regan J F, Karlin-Neumann G A, Hindson C M, Saxonov S, Colston B W. High-throughput droplet digital PCR system for absolute quantitation of DNA copy number. Anal Chem. 2011; 83(22):8604-8610. PMCID: PMC 3216358
68. McDermott G P, Do D, Litterst C M, Maar D, Hindson C M, Steenblock E R, Legler T C, Jouvenot Y, Marrs S H, Bemis A, Shah P, Wong J, Wang S, Sally D, Javier L, Dinio T, Han C, Brackbill T P, Hodges S P, Ling Y, Klitgord N, Carman G J, Berman J R, Koehler R T, Hiddessen A L, Walse P, Bousse L, Tzonev S, Hefner E, Hindson B J, Cauly T H, Hamby K, Patel V P, Regan J F, Wyatt P W, Karlin-Neumann G A, Stumbo D P, Lowe A J. Multiplexed target detection using DNA-binding dye chemistry in droplet digital PCR. Anal Chem. 2013; 85(23):11619-11627. PMID: 24180464
69. Whale A S, Huggett J F, Tzonev S. Fundamentals of multiplexing with digital PCR. Biomol Detect Quantif. 2016; 10:15-23. PMCID: PMC 5154634
70. Tewhey R, Warner J B, Nakano M, Libby B, Medkova M, David P H, Kotsopoulos S K, Samuels M L, Hutchison J B, Larson J W, Topol E J, Weiner M P, Harismendy O, Olson J, Link D R, Frazer K A. Microdroplet-based PCR enrichment for large-scale targeted sequencing. Nat Biotechnol. 2009; 27(11):1025-1031. PMCID: PMC 2779736
71. Gusel'nikova V V, Korzhevskiy D E. NeuN As a Neuronal Nuclear Antigen and Neuron Differentiation Marker. Acta Naturae. 2015; 7(2):42-47. PMCID: PMC 4463411
72. González-Acosta C A, Escobar M I, Casanova M F, Pimienta H J, BuriticáE. Von Economo Neurons in the Human Medial Frontopolar Cortex. Front Neuroanat. Frontiers; 2018; 12.
73. Allman J M, Tetreault N A, Hakeem A Y, Manaye K F, Semendeferi K, Erwin J M, Park S, Goubert V, Hof P R. The von Economo neurons in the frontoinsular and anterior cingulate cortex. Annals of the New York Academy of Sciences. 2011; 1225(1):59-71.
74. Butti C, Santos M, Uppal N, Hof P R. Von Economo neurons: Clinical and evolutionary perspectives. Cortex. 2013; 49(1):312-326.
75. Yang L, Yang Y, Yuan J, Sun Y, Dai J, Su B. Transcriptomic Landscape of von Economo Neurons in Human Anterior Cingulate Cortex Revealed by Microdissected-Cell RNA Sequencing. Cerebral Cortex. 2019; 29(2):838-851.
76. Velmeshev D, Schirmer L, Jung D, Haeussler M, Perez Y, Mayer S, Bhaduri A, Goyal N, Rowitch D H, Kriegstein A R. Single-cell genomics identifies cell type-specific molecular changes in autism. Science. 2019; 364(6441):685-689. PMCID: PMC 7678724
77. Clark I C, Delley C L, Sun C, Thakur R, Stott S L, Thaploo S, Li Z, Quintana F J, Abate A R. Targeted Single-Cell RNA and DNA Sequencing With Fluorescence-Activated Droplet Merger. Anal Chem. 2020; 92(21):14616-14623.
78. Atlas Antibodies. Cortical Layers [Internet]. 2021. Available from: https://www.atlasantibodies.com/globalassets/white-papers/023---cortical-layers-white-paper.pdf
79. Hevner R F. Layer-specific markers as probes for neuron type identity in human neocortex and malformations of cortical development. J Neuropathol Exp Neurol. 2007; 66(2):101-109. PMID: 17278994
80. Lake B B, Ai R, Kaeser G E, Salathia N S, Yung Y C, Liu R, Wildberg A, Gao D, Fung H L, Chen S, Vijayaraghavan R, Wong J, Chen A, Sheng X, Kaper F, Shen R, Ronaghi M, Fan J B, Wang W, Chun J, Zhang K. Neuronal subtypes and diversity revealed by single-nucleus RNA sequencing of the human brain. Science. 2016; 352(6293):1586-1590. PMCID: PMC5038589
81. Hodge R D, Bakken T E, Miller J A, Smith K A, Barkan E R, Graybuck L T, Close J L, Long B, Johansen N, Penn O, Yao Z, Eggermont J, Höllt T, Levi B P, Shehata S I, Aevermann B, Beller A, Bertagnolli D, Brouner K, Casper T, Cobbs C, Dalley R, Dee N, Ding S L, Ellenbogen R G, Fong O, Garren E, Goldy J, Gwinn R P, Hirschstein D, Keene C D, Keshk M, Ko A L, Lathia K, Mahfouz A, Maltzer Z, McGraw M, Nguyen T N, Nyhus J, Ojemann J G, Oldre A, Parry S, Reynolds S, Rimorin C, Shapovalova N V, Somasundaram S, Szafer A, Thomsen E R, Tieu M, Quon G, Scheuermann R H, Yuste R, Sunkin S M, Lelieveldt B, Feng D, Ng L, Bernard A, Hawrylycz M, Phillips J W, Tasic B, Zeng H, Jones A R, Koch C, Lein E S. Conserved cell types with divergent features in human versus mouse cortex. Nature. 2019; 573(7772):61-68. PMCID: PMC6919571
82. Majchrzak K, Kaspera W, Szymas J, Bobek-Billewicz B, Hebda A, Majchrzak H. Markers of angiogenesis (CD31, CD34, rCBV) and their prognostic value in low-grade gliomas. Neurol Neurochir Pol. 2013; 47(4):325-331. PMID: 23986422
83. Yamazaki T, Mukouyama Y S. Tissue Specific Origin, Development, and Pathological Perspectives of Pericytes. Front Cardiovasc Med. 2018; 5:78. PMCID: PMC6030356
84. Jurga A M, Paleczna M, Kuter K Z. Overview of General and Discriminating Markers of Differential Microglia Phenotypes. Front Cell Neurosci. Frontiers; 2020; 14.
85. Behnan J, Stangeland B, Langella T, Finocchiaro G, Tringali G, Meling T R, Murrell W. Identification and characterization of a new source of adult human neural progenitors. Cell Death Dis. 2017; 8(8):e2991. PMCID: PMC 5596556
86. Schwartz P H, Bryant P J, Fuja T J, Su H, O'Dowd D K, Klassen H. Isolation and characterization of neural progenitor cells from post-mortem human cortex. J Neurosci Res. 2003; 74(6):838-851. PMID: 14648588
87. American Type Culture Collection. Essential Neural Cell Lines [Internet]. 2019 [cited 2021 Mar. 3]. Available from: https://www.atcc.org/˜/media/PDFs/Marketing%20Material/Cell%20Biology/Neural%20Cell%20Lines.ashx
88. Chen G, Ning B, Shi T. Single-Cell RNA-Seq Technologies and Related Computational Data Analysis. Front Genet. Frontiers; 2019; 10.
89. Denisenko E, Guo B B, Jones M, Hou R, de Kock L, Lassmann T, Poppe D, Clément O, Simmons R K, Lister R, Forrest A R R. Systematic assessment of tissue dissociation and storage biases in single-cell and single-nucleus RNA-seq workflows. Genome Biol. 2020; 21(1):130.
90. Harrow J, Frankish A, Gonzalez J M, Tapanari E, Diekhans M, Kokocinski F, Aken B L, Barrell D, Zadissa A, Searle S, Barnes I, Bignell A, Boychenko V, Hunt T, Kay M, Mukherjee G, Rajan J, Despacio-Reyes G, Saunders G, Steward C, Harte R, Lin M, Howald C, Tanzer A, Derrien T, Chrast J, Walters N, Balasubramanian S, Pei B, Tress M, Rodriguez J M, Ezkurdia I, Baren J van, Brent M, Haussler D, Kellis M, Valencia A, Reymond A, Gerstein M, Guigó R, Hubbard T J. GENCODE: The reference human genome annotation for The ENCODE Project. Genome Res. 2012; 22(9):1760-1774.
91. Okazaki Y, Furuno M, Kasukawa T, Adachi J, Bono H, Kondo S, Nikaido I, Osato N, Saito R, Suzuki H, Yamanaka I, Kiyosawa H, Yagi K, Tomaru Y, Hasegawa Y, Nogami A, Schönbach C, Gojobori T, Baldarelli R, Hill D P, Bult C, Hume D A, Quackenbush J, Schriml L M, Kanapin A, Matsuda H, Batalov S, Beisel K W, Blake J A, Bradt D, Brusic V, Chothia C, Corbani L E, Cousins S, Dalla E, Dragani T A, Fletcher C F, Forrest A, Frazer K S, Gaasterland T, Gariboldi M, Gissi C, Godzik A, Gough J, Grimmond S, Gustincich S, Hirokawa N, Jackson I J, Jarvis E D, Kanai A, Kawaji H, Kawasawa Y, Kedzierski R M, King B L, Konagaya A, Kurochkin I V, Lee Y, Lenhard B, Lyons P A, Maglott D R, Maltais L, Marchionni L, Mckenzie L, Miki H, Nagashima T, Numata K, Okido T, Pavan W J, Pertea G, Pesole G, Petrovsky N, Pillai R, Pontius J U, Qi D, Ramachandran S, Ravasi T, Reed J C, Reed D J, Reid J, Ring B Z, Ringwald M, Sandelin A, Schneider C, Semple C A M, Setou M, Shimada K, Sultana R, Takenaka Y, Taylor M S, Teasdale R D, Tomita M, Verardo R, Wagner L, Wahlestedt C, Wang Y, Watanabe Y, Wells C, Wilming L G, Wynshaw-Boris A, Yanagisawa M, Yang I, Yang L, Yuan Z, Zavolan M, Zhu Y, Zimmer A, Carninci P, Hayatsu N, Hirozane-Kishikawa T, Konno H, Nakamura M, Sakazume N, Sato K, Shiraki T, Waki K, Kawai J, Aizawa K, Arakawa T, Fukuda S, Hara A, Hashizume W, Imotani K, Ishii Y, Itoh M, Kagawa I, Miyazaki A, Sakai K, Sasaki D, Shibata K, Shinagawa A, Yasunishi A, Yoshino M, Waterston R, Lander E S, Rogers J, Birney E, Hayashizaki Y, The FANTOM Consortium and the RIKEN Genome Exploration Research Group Phase I & II Team*, FANTOM Consortium:, RIKEN Genome Exploration Research Group Phase I Team:, RIKEN Genome Exploration Research Group Phase II Team:, Mouse Genome Sequencing Consortium:, Scientific management: Analysis of the mouse transcriptome based on functional annotation of 60,770 full-length cDNAs. Nature. Nature Publishing Group; 2002; 420(6915):563-573.
92. Zhang S J, Liu C J, Shi M, Kong L, Chen J Y, Zhou W Z, Zhu X, Yu P, Wang J, Yang X, Hou N, Ye Z, Zhang R, Xiao R, Zhang X, Li C Y. RhesusBase: a knowledgebase for the monkey research community. Nucleic Acids Res. Oxford Academic; 2013; 41(D1):D892-D905.
93. McLean J D, Singer S J. A General Method for the Specific Staining of Intracellular Antigens with Ferritin-Antibody Conjugates. PNAS. National Academy of Sciences; 1970; 65(1):122-128. PMID: 4189988
94. Peluso M E M, Munnia A, Tarocchi M, Giese R W, Annaratone L, Bussolati G, Bono R. Oxidative DNA damage and formalin-fixation procedures. Toxicol Res. The Royal Society of Chemistry; 2014; 3(5):341-349.
95. Dietrich D, Uhl B, Sailer V, Holmes E E, Jung M, Meller S, Kristiansen G. Improved PCR performance using template DNA from formalin-fixed and paraffin-embedded tissues by overcoming PCR inhibition. PLoS One. 2013; 8(10):e77771. PMCID: PMC 3796491
96. Douglas M P, Rogers S O. DNA damage caused by common cytological fixatives. Mutat Res. 1998; 401(1-2):77-88. PMID: 9639679
97. Kuzmin A N, Pliss A, Prasad P N. Changes in biomolecular profile in a single nucleolus during cell fixation. Anal Chem. 2014 Nov. 4; 86(21):10909-10916. PMID: 25268694
98. Katzenelenbogen Y, Sheban F, Yalin A, Yofe I, Svetlichnyy D, Jaitin D A, Bornstein C, Moshe A, Keren-Shaul H, Cohen M, Wang S Y, Li B, David E, Salame T M, Weiner A, Amit I. Coupled scRNA-Seq and Intracellular Protein Activity Reveal an Immunosuppressive Role of TREM 2 in Cancer. Cell. 2020; 182(4):872-885.e19. PMID: 32783915
99. Porichis F, Hart M G, Griesbeck M, Everett H L, Hassan M, Baxter A E, Lindqvist M, Miller S M, Soghoian D Z, Kavanagh D G, Reynolds S, Norris B, Mordecai S K, Nguyen Q, Lai C, Kaufmann D E. High-throughput detection of miRNAs and gene-specific mRNA at the single-cell level by flow cytometry. Nat Commun. 2014; 5:5641. PMCID: PMC 4256720
100. Attar M, Sharma E, Li S, Bryer C, Cubitt L, Broxholme J, Lockstone H, Kinchen J, Simmons A, Piazza P, Buck D, Livak K J, Bowden R. A practical solution for preserving single cells for RNA sequencing. Sci Rep. 2018; 8(1):2151. PMCID: PMC 5794922
101. Channathodiyil P, Houseley J. Glyoxal fixation facilitates transcriptome analysis after antigen staining and cell sorting by flow cytometry. PLoS One. 2021; 16(1):e0240769. PMCID: PMC7822327
102. Freund M, Taylor A, Ng C, Little AR. The NIH NeuroBioBank: creating opportunities for human brain research. Handb Clin Neurol. 2018; 150:41-48. PMID: 29496155
103. Schroeder A, Mueller O, Stocker S, Salowsky R, Leiber M, Gassmann M, Lightfoot S, Menzel W, Granzow M, Ragg T. The RIN: an RNA integrity number for assigning integrity values to RNA measurements. BMC Molecular Biology. 2006; 7(1):3.
104. Cattell R B. The Scree Test For The Number Of Factors. Multivariate Behavioral Research. 1966; 1(2):245-276.
105. Blondel V D, Guillaume J L, Lambiotte R, Lefebvre E. Fast unfolding of communities in large networks. J Stat Mech. 2008; 2008(10):P10008.
106. Becht E, McInnes L, Healy J, Dutertre C A, Kwok I W H, Ng L G, Ginhoux F, Newell E W. Dimensionality reduction for visualizing single-cell data using UMAP. Nat Biotechnol. 2019; 37(1):38-44.
107. Shahi P, Kim S C, Haliburton J R, Gartner Z J, Abate A R. Abseq: Ultrahigh-throughput single cell protein profiling with droplet microfluidic barcoding. Sci Rep. 2017; 7(1):44447. PMCID: PMC 5349531
108. Demaree B, Delley C L, Vasudevan H N, Peretz C A C, Ruff D, Smith C C, Abate A R. Joint profiling of proteins and DNA in single cells reveals extensive proteogenomic decoupling in leukemia. BioRxiv manuscript. 2020;
109. BioLegend, Inc. Protocol—TotalSeqTM-A Antibodies and Cell Hashing with 10x Single Cell 3′Reagent Kit v3 3.1 Protocol [Internet]. Available from: https://www.biolegend.com/en-us/protocols/totalseq-a-antibodies-and-cell-hashing-with-10x-single-cell-3-reagent-kit-v3-3-1-protocol
110. Peterson V M, Zhang K X, Kumar N, Wong J, Li L, Wilson D C, Moore R, McClanahan T K, Sadekova S, Klappenbach J A. Multiplexed quantification of proteins and transcripts in single cells. Nat Biotechnol. 2017; 35(10):936-939. PMID: 28854175
111. Stoeckius M, Hafemeister C, Stephenson W, Houck-Loomis B, Chattopadhyay P K, Swerdlow H, Satija R, Smibert P. Simultaneous epitope and transcriptome measurement in single cells. Nat Methods. 2017; 14(9):865-868.
112. Lun A T L, Riesenfeld S, Andrews T, Dao T P, Gomes T, participants in the 1st Human Cell Atlas Jamboree, Marioni J C. EmptyDrops: distinguishing cells from empty droplets in droplet-based single-cell RNA sequencing data. Genome Biol. 2019; 20(1):63. PMCID: PMC6431044
113. Zhu C, Yu M, Huang H, Juric I, Abnousi A, Hu R, Lucero J, Behrens M M, Hu M, Ren B. An ultra high-throughput method for single-cell joint analysis of open chromatin and transcriptome. Nat Struct Mol Biol. 2019; 26(11):1063-1070. PMCID: PMC 7231560
114. Chen S, Lake B B, Zhang K. High-throughput sequencing of the transcriptome and chromatin accessibility in the same cell. Nat Biotechnol. 2019; 37(12):1452-1457. PMCID: PMC6893138

I claim:

1. A method for processing cells or nuclei, comprising: a) individually encapsulating a plurality of cells or nuclei from a sample by forming a plurality of water in oil droplets in a reaction vessel, wherein the droplets comprise a bead affixed to a nucleic acid capture molecule; b) lysing the cells or nuclei and capturing RNA from the cells or nuclei with the nucleic acid capture molecule; c) removing beads with captured RNA from the droplets and reverse transcribing captured RNA; d) re-encapsulating beads to form amplification droplets; e) amplifying nucleic acid in the amplification droplets to generate amplified nucleic acid; f) removing amplified nucleic acid from the amplification droplets; and g) isolating amplified nucleic acid derived from said captured RNA by automated sorting.

2. The method of claim 1, wherein said cells comprise rare cells.

3. The method of claim 1, wherein the nuclei comprise nuclei derived from rare cells.

4. The method of any of claims 1 to 3, wherein the sample is a brain tissue sample.

5. The method of any of claims 1 to 4, wherein said droplets further comprise a lysis reagent.

6. The method of any of claims 1 to 5, wherein said reaction vessel comprises a reaction tube.

7. The method of any of claims 1 to 6, wherein said forming comprises vortexing, 8 The method of any of claim 1 to 7, wherein said capture molecule comprises a barcode.

9. The method of any of claims 1 to 8, wherein said capture molecule comprises a cDNA sequence complementary to said RNA.

10. The method of any of claim 9, wherein said cDNA is complementary to an RNA expressed in a rare cell.

11. The method of any of claim 1 to 10, comprising a plurality of different nucleic acid capture molecules affixed to one or more beads.

12. The method of claim 11, wherein said plurality of different nucleic acid capture molecules comprises a plurality of different cDNA sequences.

13. The method of claim 12, wherein said plurality of different cDNA sequences is complementary to a plurality of different RNA molecules that are each expressed in a rare cell.

14. The method of claim 13, wherein said plurality of different RNA molecules is uniquely expressed in said rare cell.

15. The method of any of claims 1 to 14, wherein said amplifying comprises PCR.

16. The method of claim 15, wherein said PCR comprises fluorescent amplicon tethering ddPCR.

17. The method of any of claims 1 to 16, wherein said isolating comprises FACS.

18. The method of any of claims 1 to 17, further comprising the step of h) analyzing said amplified nucleic acid derived from said captured RNA.

19. The method of claim 18, wherein said analyzing comprises sequencing said amplified nucleic acid derived from said captured RNA.

20. The method of any of claims 1 to 19, wherein said droplets further comprise a heat activated lysis reagent and wherein said lysing comprises heating said droplets.

21. A kit comprising one or more components useful, necessary, and/or sufficient to practice the method of any of claims 1 to 20.

22. The kit of claim 21 comprising one or more or each of: a) beads, b) nucleic acid capture molecules, c) oil, d) water, e) lysing reagent, f) reverse transcription reagents, g) nucleic acid amplification reagents, h) positive control reagents, i) negative control reagents, j) a agitation instrument, k) a sorting instrument, l) nucleic acid sequencing reagents, m) software, n) instructions, and o) a reaction vessel.

23. The kit of claim 22, wherein said nucleic acid capture molecules are affixed to said beads.

24. The kit of claim 22 or 23, wherein said nucleic acid capture molecules comprise one or more or each of: a) a barcode sequence, b) a poly-T sequence, c) a sequence complementary to said RNA, and d) a UMI sequence.

25. The kit of any of claims 22 to 24, wherein said lysing reagent is a heat activated lysing reagent.

26. The kit of any of claims 22 to 25, wherein said reverse transcription reagents comprise one or more or each of: a) a reverse transcriptase, b) a primer, c) an RNase inhibitor, d) dNTPs, e) a buffer, and f) a divalent cation.

27. The kit of any of claims 22 to 26, wherein said nucleic acid amplifications reagents comprise one or more or each of: a) a DNA polymerase, b) one or more primers, c) dNTPs, d) a buffer, e) a label, and f) a divalent cation.

28. The kit of claim 27, wherein said label comprises a fluorescent label.

29. The kit of claim 27, wherein said label is attached to a primer.

30. The kit of any of claims 22 to 29, wherein said positive control reagents comprise one or more or each of: a) a cell, b) a nucleus, and c) an RNA.

31. The kit of any of claims 22 to 30, wherein said agitation instrument comprises a vortexing instrument.

32. The kit of any of claims 22 to 31, wherein said sorting instrument comprise a FACS instrument or a MACS instrument.

33. The kit of any of claims 22 to 32, wherein said nucleic acid sequencing reagents comprise one or more or each of: a) one or more primers, b) a DNA polymerase, c) library preparation reagents, d) dNTPs, e) one or more detectable labels, and f) a buffer.

34. The kit of any of claims 22 to 33, wherein said software comprises instructions for running on a computer processor that carry out one or more or each of the functions: a) operating the agitation instrument, b) operating the sorting instrument, c) heating or cooling an instrument, d) controlling a reverse transcription reaction, e) controlling an amplification reaction, f) controlling a sequencing reaction, g) collecting data, h) storing data, i) analyzing data, and j) reporting data.

35. The kit of any of claims 22 to 34, wherein said reaction vessel is a tube.

36. The kit of claim 35, wherein said tube is microcentrifuge tube or a test tube.

37. The kit of claim 35, wherein said reaction vessel is a flask.

38. Use of a kit of any of claims 21 to 37.

39. Use of a kit of any of claims 21 to 37 for processing a cell or nuclei.

40. Use of a kit of any of claim 21 to 37 for analyzing one or more rare cells or nuclei derived from rare cells.

41. A reaction mixture comprising a reaction present during the method of any of claims 1 to 20.

42. The reaction mixture of claim 41, present during step a) of claim 1.

43. The reaction mixture of claim 41, present during step b) of claim 1.

44. The reaction mixture of claim 41, present during step c) of claim 1.

45. The reaction mixture of claim 41, present during step d) of claim 1.

46. The reaction mixture of claim 41, present during step e) of claim 1.

47. The reaction mixture of claim 41, present during step f) of claim 1.

48. The reaction mixture of claim 41, present during step g) of claim 1.
